(19) United States
(12) Patent Application Publication
(10) Pub. No.: US 2023/0147702 A1
(43) Pub. Date: May 11, 2023

(54) MICROFLUIDIC CHIPS AND MICROPHYSIOLOGICAL SYSTEMS USING THE SAME

(71) Applicant: The Board of Trustees of the Leland Stanford Junior University, Stanford, CA (US)

(72) Inventor: Wonjae Lee, Palo Alto, CA (US)

(21) Appl. No.: 17/920,541

(22) PCT Filed: Apr. 22, 2021
(86) PCT No.: PCT/US2021/028612

§ 371 Date: Oct. 21, 2022

Related U.S. Application Data
(60) Provisional application No. 63/013,903, filed on Apr. 22, 2020.

(51) Int. Cl. C12M 3/06; B01L 3/00; C12M 3/00; C12M 1/12

(52) U.S. Cl. CPC C12M 23/16; B01L 3/502761; C12M 21/08; C12M 25/02; B01L 2200/0652; B01L 2300/161

(57) ABSTRACT

Described herein is a microfluidic chip comprising a first channel in fluid communication with an adjacent second channel through a opening, wherein the height of the first channel and the second channel are chosen to generate sufficient surface tension at the opening such that a liquid injected into the first channel or the second channel is substantially confined within the first channel or the second channel, respectively, or that flow of the liquid therebetween is controlled, the surface tension producing a non-physical microfluidic barrier that limits or selectively controls passage of the liquid. Also described are in vitro microphysiological systems that use such microfluidic chips in modeling the structure and functions of human organs, such as a blood-brain barrier, and studying in vivo-like physiological responses of such organs to various investigative or therapeutic agents.

### GOVERNMENT SUPPORT CLAUSE

This invention was made with government support under contract NIH TRAINING GRANT K25 CA201545awarded by the National Institutes of Health. The government has certain rights in the invention.

### CROSS-REFERENCE TO RELATED APPLICATIONS

The present application claims the benefit of and priority to U.S. Provisional Pat. Application No. 63/013,903, filed Apr. 22, 2020 the entire disclosure of which is hereby incorporated by reference herein in its entireties for all purposes.

### BACKGROUND

### Technical Field

The present disclosure relates to an in vitro microphysiological system comprising a plurality of microfluidic channels for modeling the structure and functions of human organs, such as a blood-brain barrier, and studying in vivo-like physiological responses of such organs to various investigative or therapeutic agents.

### Description of the Related Art

Microfluidic devices featuring a plurality of microfluidic channels of various structures have been used in three-dimensional (3D) cell culture and organ-on-a-chip models to mimic the structure and functions of human biological systems and investigating physiological responses of such models to various investigative or therapeutic agents. Such microfluidics-based human microphysiological systems hold a great promise in preclinical drug development as they can provide more accurate physiological responses than traditional cell-based assays do, with a potential to replace time-consuming and costly in vivo animal tests. The human microphysiological systems utilizing such organ-on-a-chip models as central nervous system (CNS), blood-brain barrier (BBB) and neurovascular unit (NVU) enable high-throughput, real-time assessment of organ-specific therapeutic efficacy, toxicity or disease modeling, effectively reducing the cost of developing new drugs and cell therapies.

Among many organ-on-a-chip models, a microfluidic BBB model presents a particularly promising application because, despite the high complexity and cost of in vivo BBB models using animals, a high percentage of drug candidates that cleared the animal tests failed subsequently in the clinical trials. The BBB provides a homeostatic environment for the CNS and is critical for healthy brain functioning. However, the BBB’s unique barrier properties make the treatment of CNS disorders difficult, as many small- and large-molecules are restricted from entering into the brain region in quantities that are large enough to deliver a therapeutically meaningful result. Thus, it is desirable to develop a predictive, cost-effective, in vitro human BBB model that allows to monitor the transport efficacy of drugs that target the brain and examine the pathological neurovascular functions in various diseases.

Recently, stem cell therapy is emerging as a promising therapeutic treatment to restore a neurological function after an ischemic stroke. However, despite a growing number of candidate stem cell types being studied, each with their own unique characteristics, there is a lack of effective in vitro assay platforms that can systematically evaluate neurorestorative potential of the candidate cell therapy. When a dose of stem cells are transplanted into ischemic brain, its therapeutic efficacy primarily depends on the response of the NVU to these extraneous cells. Although a substantial number of studies have supported the neurorestorative potential of stem cells for stroke treatment, there have also been reports contradicting some of these observations. This could be partially because the experiments were all conducted under different conditions and/or focusing on different aspects of the complicated recovery processes. Thus, it would be desirable to develop a consistent, reproducible ischemic stroke model in the form of NVU on a microfluidic chip wherein brain microvascular endothelial cells are directed to form an intact barrier mimicking a functional human BBB and other constituent cells recapitulate in vivo-like behaviors in both healthy and ischemic conditions.

### BRIEF SUMMARY

The present disclosure provides microfluidic chips and microphysiological systems using such microfluidic chips, as well as methods of preparing and using the same, e.g., to model the structure and functions of various tissues.

In aspects the present disclosure provides a microfluidic chip, comprising a planar surface; a first channel formed on the planar surface and having a first volume defined by a first width, a first height, and a first length, the first volume extending in a first direction; and a second channel formed on the planar surface adjacent to the first channel, the second channel having a second volume defined by a second width, a second height, and a second length, the second volume extending in the first direction, the second height being greater than the first height; wherein the first channel is in fluid communication with the second channel through a first opening that extends along at least a portion of the first length, the first opening extending from the planar surface to the first height; and wherein the first height and the second height are sized to generate sufficient surface tension at the first opening such that a liquid injected into the first channel or the second channel is substantially confined within the first volume or the second volume, respectively, or that flow of the liquid therebetween is controlled, the surface tension producing a non-physical microfluidic barrier that limits or selectively controls passage of the liquid.

In some embodiments, such a microfluidic chip further comprises a third channel formed on the planar surface adjacent to the first channel, the third channel having a third volume defined by a third width, a third height, and a third length, the third volume extending in the first direction, the third height being greater than the first height; wherein the third channel is in fluid communication with the first channel through a second opening that extends along at least a portion of the first length, the second opening extending from the planar surface to the first height; and wherein the first height and the third height are sized to generate sufficient surface tension at the second opening such that a liquid injected into the first channel or the third channel is substantially confined within the first volume or the third volume, respectively, or that flow of the liquid therebetween is controlled, the surface tension producing a second non-physical microfluidic barrier that limits or selectively controls passage of the liquid.

In some embodiments, such a microfluidic chip further comprises a third channel formed on the planar surface adjacent to the second channel, the third channel having a third volume defined by a third width, a third height, and a third length, the third volume extending in the first direction, the third height being less than the second height; wherein the third channel is in fluid communication with the second channel through a second opening that extends along at least a portion of the second length, the second opening extending from the planar surface to the third height; and wherein the second height and the third height are sized to generate sufficient surface tension at the second opening such that a liquid injected into the second channel or the third channel is substantially confined within the second volume or the third volume, respectively, or that flow of the liquid therebetween is controlled, the surface tension producing a second non-physical microfluidic barrier that limits or selectively controls passage of the liquid.

Aspects of the present disclosure further include a microphysiological system, comprising a microfluidic chip described herein; and an extracellular matrix confined within the first volume of the first channel, a side wall of the extracellular matrix extending across the first opening and forming the non-physical microfluidic barrier between the first channel and the second channel.

In some embodiments, such a microphysiological system further comprises an epithelial barrier arranged in the first opening between the first channel and the second channel.

In embodiments, the microphysiological system is a vascularized tissue model. In some embodiments, the vascularized tissue model is a blood brain barrier model, a stroke model, a cardiac model, a skeletal muscle model, a liver model, a kidney model, a bone model, a skin model, an esophageal model, a gastric model, a colon model, an intestinal model, a lung model, or a pancreatic model.

Further aspects of the present disclosure include a method of preparing the microphysiological system as described herein, the method comprising: depositing an extracellular matrix precursor into the first channel of the microfluidic chip; and curing the extracellular matrix precursor to provide the extracellular matrix in the first channel.

In some embodiments, the curing the extracellular matrix precursor comprises incubating the extracellular matrix precursor, wherein the method further comprises depositing the first media into the second channel adjacent to the extracellular matrix in the first channel.

In some embodiments, the method further comprises culturing a continuous endothelial barrier in the first opening between the first channel and the second channel, wherein the first media further comprises pericytes.

Aspects of the present disclosure also include a method for screening a therapeutic agent, the method comprising: depositing the therapeutic agent in the second channel of the microfluidic chip of a microphysiological system described herein; and imaging the microfluidic chip.

Additional aspects of the present disclosure include a method for screening a therapeutic agent, the method comprising: performing a method of preparing a microphysiological system as described herein; depositing the therapeutic agent in the second channel of the microfluidic chip; and imaging the microfluidic chip.

In some embodiments, the therapeutic agent comprises a stem cell, a small molecule, or a peptide.

### BRIEF DESCRIPTION OF THE SEVERAL VIEWS OF THE DRAWINGS

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements, and have been solely selected for ease of recognition in the figures.

FIG. 1 is a perspective view of a microfluidic chip in accordance with one embodiment of the present disclosure.

FIG. 2 is a cross section view of a microfluidic chip in accordance with one embodiment of the present disclosure.

FIG. 3 is a top view of a microfluidic chip in accordance with one embodiment of the present disclosure.

FIG. 4 is a top view of a microfluidic chip in accordance with one embodiment of the present disclosure.

### DETAILED DESCRIPTION

The present disclosure relates to microfluidic chips comprising a first channel having a first height and a second channel having a second height that is greater than the first height, wherein the first channel is in fluid communication with the second channel through a first opening, and wherein the first height and the second height are sized to generate sufficient surface tension at the first opening such that a liquid injected into the first channel or the second channel is substantially confined within the first channel or the second channel, or that flow of the liquid therebetween is controlled, the surface tension producing a non-physical microfluidic barrier that limits or selectively controls passage of the liquid. Also described are microphysiological systems comprising such microfluidic chips, as well as methods of making and using the same.

The microchips and microphysiological systems of the present disclosure have one or more of the following advantages:
(a) There are no physical structures (e.g., capillary pressure barriers or membranes) in the opening between adjacent channels. This not only allows for unobstructed interaction at the interface between the channels, but also allows an epithelial layer on a sidewall of an extracellular matrix in the opening between adjacent channels to be continuous and intact. Capillary pressure barriers may hinder cellular interaction at the interface between the channels and may cause physical defects in an epithelial layer. Such defects may provide therapeutic agents with a shortcut through the epithelial layer. Thus, the continuous and intact epithelial layer results in consistent assessment of therapeutic agents.
(b) The described MPS allow for personalized screening models using patient-derived cells and simulating the unique pathophysiological condition of individual patients.
(c) The in vitro ischemic stroke model described shows induced inflammation and deterioration in tissue integrity and endogenous neuroprotection and tissue remodeling.

Prior to setting forth this disclosure in more detail, it may be helpful to an understanding thereof to provide definitions of certain terms to be used herein. Additional definitions are set forth throughout this disclosure.

As used herein, the term “channel” refers to a microfluidic channel, i.e., an enclosed passage formed on a layer. A channel has a volume defined by a width, a height, and a length, at least one of which being in the sub-millimeter range. As is understood, the term channel encompasses linear channels, as well as channels with portions extending in more than one direction (i.e., channels with bends or curves) and branched channels. A channel typically comprises an inlet through which a volume of liquid can be injected. Channels optionally also comprise outlets or vents. The volume enclosed by a microfluidic channel is typically in the microliter or sub-microliter range. In some embodiments, the cross-sectional dimension of a channel is less than 1 millimeter, less than 500 micrometers, less than 100 micrometers, less than 50 micrometers, or less than 25 micrometers.

“Microphysiological system,” also known as “organ-on-chip”, refers to a microfabricated platform designed to model functional units of organs in vitro. Microphysiological systems enable close contact between different cell types (e.g., between epithelium and vascular endothelium), while producing spatiotemporal gradients of chemicals and mechanical strain to mimic organ functions.

As used herein, “biological tissue” refers to a collection of functionally interconnected cells that are to be cultured and/or assayed using the methods described herein. The cells may be a cell aggregate, or a particular tissue sample from a patient. For example, “biological tissue” encompasses organoids, tissue biopsies, tumor tissue, resected tissue material and embryonic bodies.

The term “stem cell” as used herein, refers to totipotential or pluripotential precursor cells capable of generating a variety of mature human cell lineages. In other words, stem cells are undifferentiated or partially differentiated cells that can differentiate into various types of cells.

As used herein, the term “small molecule” refers to a low molecular weight (< 900 daltons) organic compound that may have some biological activity, with a size on the order of 1 nanometer (nm).

“Peptide” refers to a polymer of amino acid residues. Peptides include naturally occurring amino acid polymers and non-naturally occurring amino acid polymers, as well as amino acid polymers in which one or more amino acid residues is an artificial chemical mimetic of a corresponding naturally occurring amino acid.

As used herein, “amino acid” refers to naturally occurring amino acids and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group (e.g., homoserine, norleucine, methionine sulfoxide, and methionine methyl sulfonium). Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

A “probe” is a group of atoms or molecules that can be used to detect an analyte. In response to the analyte, a measureable property of the probe changes. A “luminescent probe” refers to a probe or molecule that emits light. Types of luminescent probes include bioluminescent, chemiluminescent, electrochemiluminescent, electroluminescent, and photoluminescent. The probe may be luminescent on its own or the resulting luminescence may the consequence of a chemical or enzymatic reaction involving the luminescent probe. Alternatively, the probe may be fluorescent.

“Fluorescent” refers to a molecule which is capable of absorbing light of a particular frequency and emitting light of a different frequency.

The term “polymer” refers to a material comprising a macromolecule composed of repeated subunits. Each subunit is referred to as a monomer. Polymers may be natural, semisynthetic, or synthetic.

The use of the words “optional” or “optionally” means that the subsequently described event or circumstances may or may not occur, and that the description includes instances wherein the event or circumstance occurs and instances in which it does not.

As used herein, the term “about” means ± 20%, ± 10%, ± 5% or ± 1% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives.

Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to,” such that recitation of items in a list is not to the exclusion of other like items that may also be useful in the materials, compositions, devices, and methods of this technology. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Similarly, the terms “can” and “may” and their variants are intended to be non-limiting, such that recitation that an embodiment can or may comprise certain elements or features does not exclude other embodiments of the present technology that do not contain those elements or features. Furthermore, the particular features, structures, or characteristics may be combined in any suitable manner in one or more embodiments.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated.

In the following description, certain specific details are set forth in order to provide a thorough understanding of various embodiments of this disclosure. However, one skilled in the art will understand that the disclosure may be practiced without these details.

### Microfluidic Chips

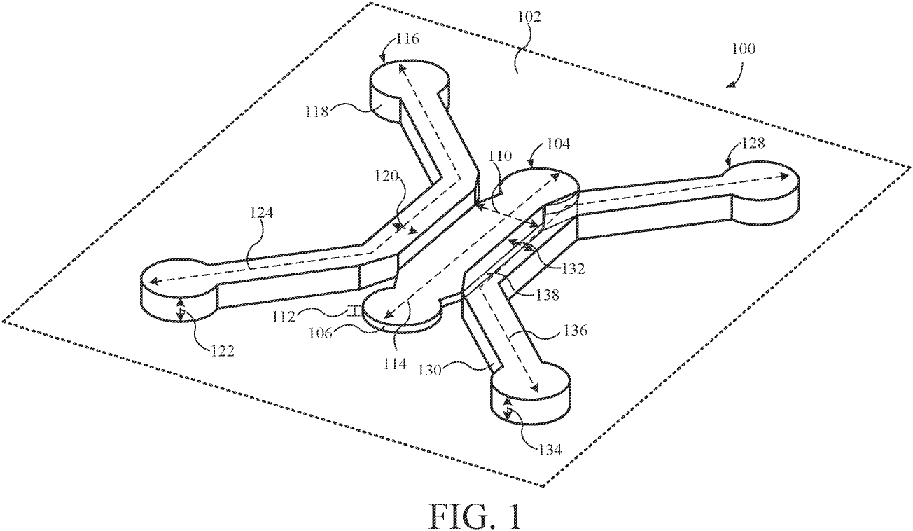

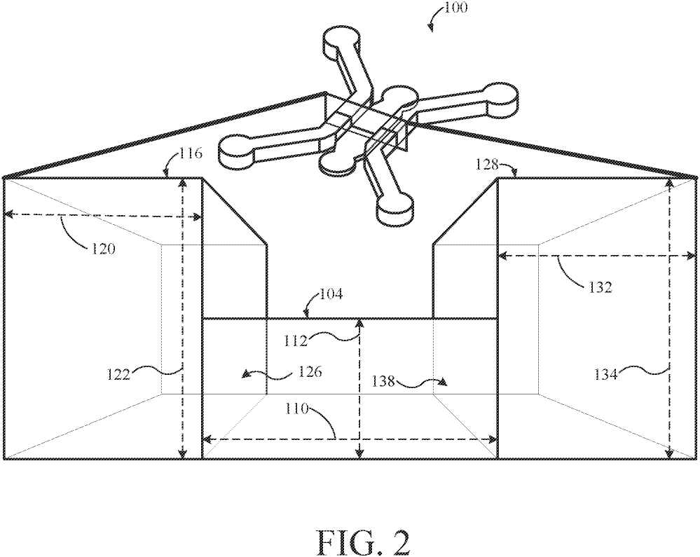

As noted above, the present disclosure provides microfluidic chips that comprise channels having different heights. A perspective view of an embodiment of a microfluidic chip of the present disclosure is shown in FIG. 1. A cross section of the microfluidic chip of FIG. 1, is shown in FIG. 2.

The microfluidic chip 100 comprises a planar surface 102 on which a first channel 104 is formed. The first channel 104 has a first volume 106 that extends in a first direction. The first volume 106 is defined by a first width 110, a first height 112, and a first length 114. A second channel 116 is formed on the planar surface 102 adjacent to the first channel 104. The second channel 116 has a second volume 118 that extends in the first direction and is defined by a second width 120, a second height 122, and a second length 124. As illustrated, the second height 122 is greater than the first height 112.

The first channel 104 is in fluid communication with the second channel 116 through a first opening 126 (shown in FIG. 2) that extends along at least a portion of the first length 114, the first opening 126 extending from the planar surface 102 to the first height 112. In various embodiments, the first opening 126 extends along 25% to 75% of the first length 114. In particular embodiments, the first opening extends along about 50% of the first length.

In the microfluidic chips of the present disclosure, the first opening 126 is unobstructed. In other words, there is no physical barrier, such as a capillary pressure barrier (e.g., a phaseguide, rim, ridge, pillars, and the like) or membrane, in the first opening 126. Instead, the first height 112 and the second height 122 are sized to generate sufficient surface tension at the first opening 126 such that a liquid injected into the first channel or the second channel is substantially confined within the first volume or the second volume, respectively, or that flow of the liquid therebetween (i.e., between the first and second volumes) is controlled, the surface tension producing a non-physical microfluidic barrier that limits or selectively controls passage of the liquid. In other words, the difference between the first height and the second height provides enough surface tension that a liquid injected into the first channel does not spread into the second channel through the opening.

Accordingly, the present disclosure provides a microfluidic chip, comprising: a planar surface; a first channel formed on the planar surface and having a first volume defined by a first width, a first height, and a first length, the first volume extending in a first direction; and a second channel formed on the planar surface adjacent to the first channel, the second channel having a second volume defined by a second width, a second height, and a second length, the second volume extending in the first direction, the second height being greater than the first height; wherein the first channel is in fluid communication with the second channel through a first opening that extends along at least a portion of the first length, the first opening extending from the planar surface to the first height; and wherein the first height and the second height are sized to generate sufficient surface tension at the first opening such that a liquid injected into the first channel or the second channel is substantially confined within the first volume or the second volume, respectively, or that flow of the liquid therebetween (i.e., between the first volume and the second volume) is controlled, the surface tension producing a non-physical microfluidic barrier that limits or selectively controls passage of the liquid.

Sufficient surface tension is provided when the atmospheric pressure is less than the capillary pressure in the microfluidic channel. As is understood, the capillary pressure in a microfluidic channel (P_{c}) can be calculated based on the following equation:

$${P}_{c} = γ ⋅ \left(\frac{cos {θ}_{l e f t} + cos {θ}_{r i g h t}}{w} + \frac{cos {θ}_{b o t t o m} + cos {θ}_{t o p}}{h}\right)$$

Where:
γ = the surface tension of liquid in the microfluidic channel
h = the channel height
w = the channel width
θ_{bottom}, θₜₒₚ, θ_{left}, θ_{right} = the bottom, top, left, and right contact angles of the liquid injected into the microfluidic channel, respectively.

The height difference between neighboring channels allows for Pc to be higher than the atmospheric pressure, Pa, according to the following equation:

$${P}_{c} - {P}_{a} = γ \cdot \left(\frac{cos {θ}_{l e f t} + cos {θ}_{r i g h t}}{w} + \frac{cos {θ}_{b o t t o m} + cos {θ}_{t o p}}{h}\right) - {P}_{a} > 0$$

In embodiments, a microfluidic chip of the present disclosure comprises at least three channels with a first opening between the first and second channels, and a second opening between the first and third channels (discussed in further detail below). In such a configuration, there is no left or right surface with respect to the middle (i.e., first) channel. Accordingly, θ_{left} = θ_{right} = 0.

$${p}_{c} - {p}_{a} = γ ⋅ \left(\frac{2}{w} + \frac{cos {θ}_{b o t t o m} + cos {θ}_{t o p}}{h}\right) - {p}_{a} > 0$$

Pₐ= atmospheric pressure

In various embodiments, the first height 112 is at least 90% of the second height 122. In some embodiments, the first height 112 is at least 80% of the second height 122. In some embodiments, the first height 112 is at least 70% of the second height 122. In some embodiments, the first height 112 is at least 60% of the second height 122. In some embodiments, the first height 112 is at least 50% of the second height 122. In further embodiments, the first height 112 is at least 25% of the second height 122. In particular embodiments, the first height 112 is at least 10% of the second height 122. It is preferable to minimize the difference between the first and second heights to the extent possible in order to maximize the size of the opening, and thus the size of the contact area between the channels.

The specific dimensions of the respective channels may be altered for a particular purpose or design. In embodiments, the first height ranges from 10 micrometers (µm) to 900 µm. In some embodiments, the first height ranges from 10 µm to 720 µm. In some embodiments, the first height ranges from 75 micrometers µm to 720 µm. In some embodiments, the first height ranges from 10 µm to 450 µm. In some embodiments, the first height ranges from 75 µm to 450 µm. In a particular embodiment, the first height is about 100 µm. In embodiments, the first width ranges from 1.25 millimeters (mm) to 2.5 mm. In a particular embodiment, the first width is about 1.5 mm. In other embodiments, the first width is about 2 mm. In embodiments, the second height ranges from 300 µm to 1000 µm. In embodiments, the second height ranges from 300 µm to 800 µm. In further embodiments, the second height ranges from 300 µm to 500 µm. In a particular embodiment, the second height is about 400 µm. In another particular embodiment, the second height is about 700 µm. In some embodiments, the second width ranges from 0.75 mm to 1.5 mm. In a particular embodiment, the second width is about 1 mm. In a specific embodiment, the first height is about 100 µm, the first width is about 2 mm, the second height is about 400 µm, and the second width is about 1 mm.

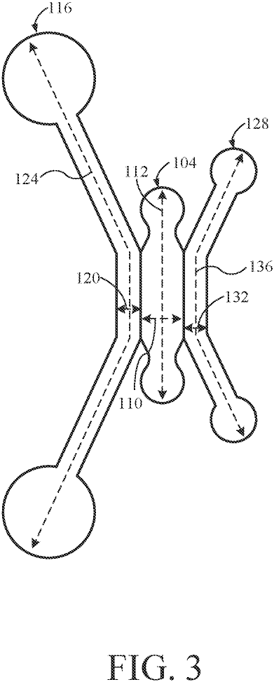

The various channels may have any suitable length. As the length of the channels does not impact the surface tension produced in an opening between channels, other factors may be considered in selecting channel length. For example, the length of a particular channel may be chosen, in part, in order to minimize contamination between channels (e.g., by increasing the distance between inlet(s), outlet(s), or vent(s) of adjacent channels). As illustrated in FIG. 3, the second length 124 may be significantly longer than the first length 114 in order to space the ends of the channels apart. In various embodiments, the first length ranges from 5 mm to 10 mm. In a particular embodiment, the first length is about 8 mm. In some embodiments, the second length ranges from 1.5 centimeters (cm) to 2.5 cm. In a particular embodiment, the second length is about 2 cm. In some embodiments, the length of the second channel ranges from 0.5 cm to 1.5 cm. In a particular embodiment, the second length is about 1 cm. In a specific embodiment, the first length is about 8 mm and the second length is about 2 cm. In microfluidic chips that comprise more than two channels, e.g. three channels, the length of the third channel may be the same as the length of the second channel. In other embodiments, the length of the third channel is longer than the length of the second channel. In further embodiments, the length of the third channel is shorter than the length of the second channel. In still further embodiments, the length of the second and/or third channel(s) is less than the length of the first channel.

As noted above, each of the microfluidic channels may include an inlet, as well as an outlet or vent, as required for a particular use. In various embodiments, each microfluidic channel includes an inlet and an outlet in order to facilitate filling, emptying, and perfusion of a liquid. Such inlets and outlets are generally formed as an aperture in the cover layer, but other configurations are also contemplated.

As shown in FIG. 1 and FIG. 2, a microfluidic chip of the present disclosure may comprise a third channel 128 adjacent to the first channel 104. The third channel 128 has a third volume 130 that extends in the first direction and is defined by a third width 132, a third height 134, and a third length 136. In the illustrated configuration, the third channel 128 in fluid communication with the first channel 104 through a second opening 138 that extends along at least a portion of the first length 114. The second opening 138 extends from the planar surface 102 to the first height 112. As shown, the third height 134 is greater than the first height 112. Additionally, the first height 112 and the third height 134 are sized to generate sufficient surface tension at the second opening 138 such that a liquid injected into the first channel 104 or the third channel 128 is substantially confined within the first volume 106 or the third volume 130, respectively, or that flow of the liquid therebetween (i.e., between the first volume and the third volume) is controlled, the surface tension producing a second non-physical microfluidic barrier that limits or selectively controls passage of the liquid. The first height 112 and the third height 134 are sized in a manner similar to that described above with regard to the first height 112 and the second height 122. In some embodiments, the second height 122 and the third height 134 are the same.

In another embodiment, a third channel is formed on the planar surface adjacent to the second channel. The third channel has a third volume that extends in the first direction and is defined by a third width, a third height, and a third length. In this configuration, the third channel in fluid communication with the second channel through a second opening that extends along at least a portion of the third length. The second opening extends from the planar surface to the third height, which is less than the second height. Additionally, the second height and the third height are sized to generate sufficient surface tension at the second opening such that a liquid injected into the second channel or the third channel is substantially confined within the second volume or the third volume, respectively, or that flow of the liquid therebetween (i.e., between the second volume and the third volume) is controlled, the surface tension producing a second non-physical microfluidic barrier that limits or selectively controls passage of the liquid. Thus, the second height and the third height are sized in a manner similar to that described above with regard to the first height and the second height. In some embodiments, the first height and the third height are the same.

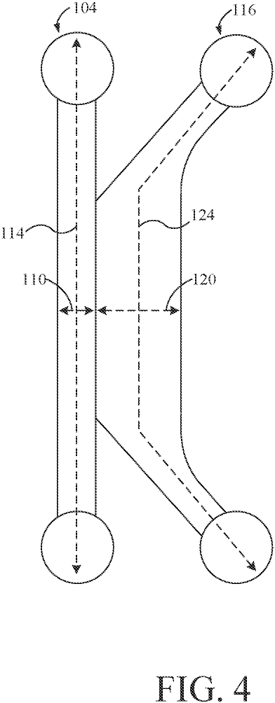

Although the microfluidic chip of FIG. 1 and FIG. 2 is illustrated with three channels, it will be clear from the foregoing description that a microfluidic chip of the present disclosure may have any suitable number of channels. An exemplary two-channel microfluidic chip is shown in FIG. 4. Any suitable number (e.g., two, three, four, five, six, etc.) and arrangement of channels may be used, provided that the height difference between adjacent channels provides sufficient surface tension in the respective opening, as discussed above with respect to the first channel and the second channel.

Further, a microfluidic chip of the present disclosure may be in a multi-array format, also referred to as a multi-well format, to enable its use in in-vitro cell-based assays, pharmaceutical screening assays, toxicity assays, and the like, such as in a high-throughput screening format. For example, a multi-array culture plate with 6, 12, 24, 48, 96, 384, or 1536 sample wells (e.g., multi-channel arrangements for testing a single sample) arranged in a rectangular matrix. In embodiments, the microfluidic chip is compatible with one or more dimensions of the standard ANSI/SLAS microtiter plate format.

The microfluidic chips of the present disclosure may be constructed using any suitable techniques, such as photolithography techniques, hot- embossing techniques, soft embossing techniques, etching techniques, replication molding or injection molding techniques.

### Microphysiological Systems and Methods of Making the Same

Also described herein are microphysiological systems (MPS) comprising a microfluidic chip of the disclosure. Such MPS comprise an extracellular matrix confined within the first volume of the first channel. A sidewall of the extracellular matrix extends across the first opening and forms the non-physical microfluidic barrier between the first channel and the second channel.

The extracellular matrix may be any suitable gel on the surface of which epithelial cells can be cultured. For example, the extracellular matrix may comprise synthetic polymers or a natural polymers (e.g., biopolymers), including hydrogel, agarose, gelatin, dextran, chitosan, silica gel and the like. In embodiments, the extracellular matrix comprises a basement membrane extract, human or animal tissue or cell culture-derived extracellular matrices, animal tissue-derived extracellular matrices, synthetic extracellular matrices, hydrogels, collagen, soft agar, egg white, or a combination thereof.

The extracellular matrix may also comprise growth and/or differentiation substrates, such as collagen, collagen I, collagen IV, fibronectin, laminin, vitronectin, D-lysine, entactin, heparan sulfate proteoglycans, or combinations thereof. In another embodiment, the matrix may comprise laminin, collagen IV, entactin, and heparin sulfate proteoglycan. In some such embodiments, the matrix also contains growth factors, matrix metalloproteinases (collegenases), other proteinases (plasminogen activators), or a combination thereof. In another embodiment, the extracellular matrix comprises a basement membrane extract, an extracellular matrix component, collagen, collagen I, collagen IV, fibronectin, laminin, vitronectin, D-lysine, entactin, heparan sulfide proteoglycans, or a combination thereof.

In embodiments, the extracellular matrix comprises a hydrogel. As used herein a “hydrogel” is a three-dimensional network of crosslinked hydrophilicpolymer chains. Hydrogels used for cell culture may include natural and/or synthetic materials. Suitable natural hydrogels for cell culture may comprise proteins and extracellular matrix components such as collagen, fibrin, hyaluronic acid, or Matrigel, as well as materials derived from other biological sources such as chitosan, alginate, or silk fibrils. Suitable synthetic hydrogels may be formed of non-natural molecules such as poly(ethylene glycol) (PEG), poly(vinyl alcohol), and poly(2-hydroxy ethyl methacrylate).

In some embodiments, the extracellular matrix comprises a basement membrane extract. Basement membranes are thin extracellular matrices comprising proteins and proteoglycans, and which underlie epithelial cells in vivo. Epithelial cells work in conjunction with a basement membrane to form a solid barrier to protect the internal vital activity.

The extracellular matrix is provided to the microfluidic chip in the form of an extracellular matrix precursor. As used herein “extracellular matrix precursor” refers to a liquid polymer or pre-polymer that, once cured, provides an extracellular matrix as described herein.

After the extracellular matrix precursor is provided, it is at least partially cured (i.e., gelled), prior to introduction of a further liquid into an adjacent channel. In some embodiments, the extracellular matrix is fully cured prior to introducing a liquid into the adjacent channel. Accordingly, a method of preparing an MPS of the present disclosure comprises depositing an extracellular matrix precursor into the first channel of the microfluidic chip; and curing the extracellular matrix precursor to provide the extracellular matrix in the first channel. The conditions used to cure the extracellular matrix precursor will vary depending on the extracellular matrix used. Any method of curing suitable for the particular extracellular matrix precursor may be used. In embodiments, curing the extracellular matrix precursor comprises allowing the extracellular matrix precursor to set. In particular embodiments, curing the extracellular matrix precursor comprises incubating the extracellular matrix precursor while it sets. The extracellular matrix precursor may be incubated for example, at about 37° C. In other embodiments, curing the extracellular matrix precursor comprises incubating the extracellular matrix precursor with a crosslinking agent. In further embodiments, curing the extracellular matrix precursor comprises ionic polymerization, thermal polymerization, or photopolymerization.

In embodiments, the extracellular matrix also comprises cells (e.g., from a biological tissue sample). For example, the biological tissue may comprise an organoid, tissue biopsy, tumor tissue, resected tissue material, or embryonic body. The biological tissue sample may comprise cells obtained from, derived from, or exhibiting a phenotype associated with a particular tissue or organ, for example neural cells, cardiac cells, hepatic cells, renal cells, skeletal muscle cells, bone cells, skin cells, esophageal cells, intestinal cells, gastric cells, colon cells, lung cells, or pancreatic cells. In particular embodiments, the cells are neural cells. In some such embodiments, the neural cells comprise human induced pluripotent stem cell-derived neural progenitor cells, astrocytes, microglia, or combinations thereof. The biological tissue sample may be derived from healthy or diseased tissue. Other suitable cells include human iPSC-derived cells, embryonic stem cells, and other pluripotent cells, progenitor cells, differentiable cells, and the like. For example, neurons, endothelial cells, epithelial cells, astrocytes, pericytes, cardiomyocytes, skeletal muscle cells, hepatocytes, fibroblasts, osteocytes, and the like.

In some embodiments, cells are deposited in the extracellular matrix precursor before it is cured. In some such embodiments, the cells are present in the extracellular matrix precursor before it is deposited into the first channel. In other embodiments, the cells are introduced into or onto the extracellular matrix.

As noted above, the extracellular matrix precursor is cured prior to introduction of a further liquid into an adjacent channel. Thus, in embodiments, a method of preparing an MPS of the disclosure comprises curing the extracellular matrix precursor, wherein media is deposited into the second channel adjacent to the extracellular matrix in the first channel. Accordingly, in particular embodiments, a method of preparing an MPS of the disclosure comprises curing the extracellular matrix precursor by incubating the extracellular matrix precursor, wherein media is deposited into the second channel adjacent to the extracellular matrix in the first channel.

In some such embodiments, the media is confined within the volume of a channel (e.g., the second channel) adjacent to a channel comprising an extracellular matrix (e.g., the first channel). For example, in some embodiments, the media is confined within the second volume of the second channel. In embodiments, the microfluidic chip comprises a third channel adjacent to the first channel and the third height is greater than the first height. In some such embodiments, the MPS further comprises a second media confined within the third volume of the third channel.

In other embodiments, the microfluidic chip comprises a third channel adjacent to the second channel and the third height is less than the second height. In some such embodiments, the MPS further comprises a second extracellular matrix confined within the third volume of the third channel.

In an MPS of the present disclosure, an epithelial layer, also referred to as an epithelial barrier, is arranged in an opening between adjacent channels. For example, in some embodiments an MPS comprises an epithelial barrier arranged in the first opening between the first channel and the second channel.

In embodiments, the epithelial barrier (e.g., endothelial barrier) is continuous. In some embodiments, the epithelial barrier is continuous if there is 75% confluence. As used herein “confluence” is used as an estimate of the proportion of the sidewall of an extracellular matrix in the opening between adjacent channels that is covered by adherent epithelial cells. In embodiments, the epithelial barrier is continuous if there is 85% confluence. In embodiments, the epithelial barrier is continuous if there is 90% confluence. In embodiments, the epithelial barrier is continuous if there is 95% confluence. In specific embodiments, an epithelial barrier is considered continuous if it is confluent (i.e., about 100% of the sidewall of the extracellular matrix is covered by adherent cells).

Any suitable epithelial cells may be used. As used herein, an “epithelial cell” refers to a cell of epithelial origin, or a cell that is differentiated into a state in which it expresses markers identifying the cell as an epithelial cell.

The cells may be animal cells (e.g., epithelial cells, cells from an epithelial cell line, epithelial primary cells). For example, the cells may be from a mammal (e.g., mouse, rat, canine, feline, bovine, equine, porcine, non-human primate, and human). In particular embodiments, the cells are human cells (e.g., epithelial cells, cells from an epithelial cell line, epithelial primary cells). Examples of epithelial cells that may be used include prostate cells, mammary cells, hepatocytes, pancreatic islet cells (e.g., beta cells), pulmonary epithelial cells, kidney cells, bladder cells, stomach epithelial cells, large and small intestinal epithelial cells, urethral epithelial cells, testicular epithelial cells, ovarian epithelial cells, cervical epithelial cells, thyroid cells, parathyroid cells, adrenal cells, thymus cells, gall bladder cells, and pituitary cells. In addition, transformed cells or established cell lines can also be used. As used herein, the term “cell line” refers to continuously growing or immortalized cells. In other words, a cell line is a population of cells from a multicellular organism which would normally not proliferate indefinitely but, due to mutation, have evaded normal cellular senescence and instead can keep undergoing division.

In embodiments, the epithelial barrier comprises endothelial cells. An “endothelial cell” is a cell of endothelial origin, or a cell that is differentiated into a state in which it expresses markers identifying the cell as an endothelial cell. In some embodiments, the epithelial barrier is an endothelial barrier. In some embodiments, the endothelial cells are derived from stem cells. In particular embodiments, the endothelial cells are derived from induced pluripotent stem cells. In some embodiments, the endothelial cells comprise blood outgrowth endothelial cells. The epithelial cells (e.g., endothelial cells) forming the epithelial barrier may be obtained from or derived from the same subject from which the biological tissue sample was obtained. In particular embodiments, the endothelial barrier comprises Brain Microvascular Endothelial Cells (BMEC). In specific embodiments, the endothelial barrier comprises human BMEC.

Embodiments of the methods of preparing an MPS of the disclosure comprise culturing a continuous endothelial barrier in the first opening between the first channel and the second channel. As noted above, such an epithelial barrier (e.g., endothelial barrier) is cultured on the sidewall of the extracellular matrix after the extracellular matrix precursor has been cured and after the media has been deposited into the adjacent channel. In some embodiments, the epithelial cells are deposited in a media in a channel adjacent to the epithelial barrier (e.g., the second channel) and the microfluidic chip is tilted such that the epithelial cells settle on the sidewall of the extracellular matrix.

The media contained in the channel adjacent to the channel containing the extracellular matrix is selected according to the function of the channel. For example, the media in a channel adjacent to the epithelial barrier (e.g., the second channel) is typically a growth medium that provides nutrients and oxygen after the epithelial cells are injected. Additionally, the media contained in such a channel may be changed or replaced at various times.

In some embodiments, the media in the channel adjacent to the epithelial barrier (e.g., the second channel) further comprises pericytes. In other words, in some embodiments, pericytes are contained in the second channel. In such embodiments, the media in the second channel may comprise a combination of endothelial cell medium and pericyte medium.

In embodiments, flow is simulated in at least one channel. In various embodiments, flow is simulated by rocking the microfluidic chip in the first direction. In such embodiments, flow is not simulated in the microfluidic chip until after the epithelial barrier is established (e.g., the epithelial barrier has 75%, 85%, 90%, 95%, or 100% confluence).

As will be appreciated, the MPS of the present disclosure are well suited to be used as vascularized tissue models in which a media containing channel (e.g., the second channel) acts as a ‘blood’ channel and an extracellular matrix containing channel (e.g., the first channel) acts as a tissue (e.g., brain, heart, liver, kidney, skeletal muscle, bone, skin, esophagus, intestine, stomach, colon, lung, pancreas, etc.) channel. Accordingly, in embodiments, the MPS of the present disclosure are a vascularized tissue model. In some embodiments, the vascularized tissue model is a blood brain barrier model or a stroke model. In further embodiments, the vascularized tissue model is a cardiac model, a skeletal muscle model, a liver model, a kidney model, a bone model, a skin model, an esophageal model, a gastric model, a colon model, an intestinal model, a lung model, or a pancreatic model.

In particular embodiments, a microfluidic chip of the disclosure, as applied to a human blood-brain-barrier (BBB), comprises three channels: a “blood-side” channel (i.e., a second channel, as discussed above) through which a bloodstream is simulated; a middle “brain” channel (i.e., a first channel, as discussed above) in which neural cells form a native 3D structure in a hydrogel matrix; and a “cerebrospinal fluid (CSF)-side” channel (i.e., a third channel, as discussed above) that provides an additional fluidic access to the neural cells in the “brain” channel. In a preferred embodiment, the middle “brain” channel is configured to be lower in height to generate the surface tension between a upper surface and a lower surface of the channels, which operates to stably contain the liquid hydrogel precursor in the “brain” channel. This embodiment of a BBB model can generate a continuous and physically intact endothelial barrier which provides a well-defined boundary between the “blood-side” channel and the “brain” channel.

### Methods of Use

The present disclosure also provides methods of using the MPS described herein. In particular, the microfluidic devices of the present disclosure and the MPS produced thereon, may be used in methods of assaying or testing various human biological systems using these chips and models, such as BBB, NVU, CNS, blood vessels, livers, kidneys, intestines, or cancer tumors. Further, the MPS may be used in 3D cell culture, co-culture, migration studies, cytotoxicity studies, and various other cell assays. In particular embodiments, the MPS of the present disclosure is a vascularized tissue model. In various embodiments, the vascularized tissue model is a cardiac model, a skeletal muscle model, a liver model, a kidney model, a bone model, a skin model, an esophageal model, a gastric model, a colon model, an intestinal model, a lung model, or a pancreatic model.

For example, MPS of the present disclosure may be used in methods of screening therapeutic agent(s). In such methods, an MPS of the present disclosure is prepared as described above. After the epithelial barrier is established, a therapeutic agent is deposited in the media in a channel adjacent to the epithelial barrier (e.g., the second channel). After a suitable time period, the microfluidic chip is analyzed. In some embodiments, the analysis comprises imaging studies. In some embodiments, the cells or tissues in the extracellular matrix may be lysed and gene expression may be analyzed. Such methods of screening may be used for any suitable therapeutic agent(s). For example, the therapeutic agent may be a stem cell, a small molecule, or a peptide.

By way of example, an MPS of the disclosure may be prepared as described above (e.g., by (1) depositing an extracellular matrix precursor comprising a biological tissue sample in a first channel, (2) curing the extracellular matrix precursor, (3) depositing a media comprising endothelial cells and pericytes in a second channel adjacent to the first channel, and (4) culturing a continuous endothelial barrier on a sidewall of the extracellular matrix in the opening between the first and second channel). After the MPS is prepared and the endothelial barrier is established, a therapeutic agent is deposited into the second channel. After a suitable period of time, the MPS is analyzed. For example, the microfluidic chip may be stained and imaged. Alternatively, or in addition, the extracellular matrix and embedded cells are lysed and gene expression is analyzed. Further, media from the third channel may be removed an analyzed, for example, for concentrations of the therapeutic agent, a metabolite, or the like. Such methods may be repeated in serial or in parallel with multiple therapeutic agent(s). In some embodiments, several replicates are performed for each therapeutic agent.

In some embodiments, the methods of screening are specific to a particular patient. In such embodiments, the results of the screen may be used in treatment decisions. In particular embodiments, the cells used in an MPS of the present disclosure are patient-derived cells. In some such embodiments, the unique pathophysiological condition of individual patients is also simulated in the MPS.

In various embodiments, portions of the MPS are stained prior to imaging. In some embodiments, a probe is introduced into the MPS prior to imaging. Any suitable stain (e.g., CD31, von Willebrand factor, etc.) or probe (e.g., luminescent probe) may be used in accordance with known protocols. Imaging studies may be used, for example, to assess neural degeneration, to track stem cell infiltration, record cytosolic calcium oscillation in the neurons in an ischemic stroke model, to assess the status of the endothelial barrier, and the like.

### Kits

The present disclosure further provides for kits for use in preparing an MPS or using an MPS (e.g., in a method of screening therapeutic agent(s)) as described herein. Kits of the present disclosure comprise a microfluidic chip as discussed above. In some embodiments, a kit of the present disclosure also comprises an extracellular matrix precursor or a component thereof. In a particular embodiment, a kit of the present disclosure comprises a microfluidic chip and a pro-angiogenic compound.

Such kits can further comprise one or reagents, assay controls, or other supplies necessary for evaluation of therapeutic agent(s), such as syringes, ampules, vials, tubes, tubing, facemask, a needleless fluid transfer device, an injection cap, sponges, sterile adhesive strips, Chloraprep, gloves, and the like. Variations in contents of any of the kits described herein can be made.

The kits can further comprise written instructions for using the kit in the methods disclosed herein. In various embodiments, the written instructions may include instructions regarding preparation of the reagents; appropriate reference levels to interpret results associated with using the kit; proper disposal of the related waste; and the like. The written instructions can be in the form of printed instructions provided within the kit, or the written instructions can be printed on a portion of the container housing the kit. Written instructions may be in the form of a sheet, pamphlet, brochure, CD-ROM, or computer-readable device, or can provide directions to locate instructions at a remote location, such as a website. The written instructions may be in English and/or in a national or regional language.

### EXAMPLE 1

### Microphysiological Stroke Model for Systematic Evaluation of Neurorestorative Potential of Stem Cell Therapy

Stem cell therapy is emerging as a promising treatment option to restore a neurological function after ischemic stroke. Despite the growing number of candidate stem cell types, each with unique characteristics, there is a lack of experimental platform to systematically evaluate their neurorestorative potential. When stem cells are transplanted into ischemic brain, the therapeutic efficacy primarily depends on the response of the neurovascular unit (NVU) to these extraneous cells. An ischemic stroke microphysiological system (MPS) with a functional NVU on a microfluidic chip was developed. The new chip design facilitated the incorporated cells to form a functional blood-brain barrier (BBB) and restore their in vivo-like behaviors in both healthy and ischemic conditions. The MPS was used to track the transplanted stem cells and characterize their neurorestorative behaviors reflected in gene expression levels. Each type of stem cells showed unique neurorestorative effects, primarily through supporting the endogenous recovery, rather than through direct cell replacement. The recovery of synaptic activities, critical for neurological function, was more tightly correlated with the recovery of the structural and functional integrity in NVU, rather than with the regeneration of neurons itself.

### Chip Design for the Reconstruction of Functional BBB

The key advantage of an in vitro model is the capacity of the real-time monitoring of the cell behaviors. The microfluidic chip has three channels; the ‘blood-side’ channel through which the bloodstream is simulated, the ‘brain’ channel in which the neural cells form a native 3D structure in a hydrogel matrix, and the ‘cerebrospinal fluid (CSF)-side’ channel which provides an additional access to the neural cells in the ‘brain’ channel. The samples were prepared by deploying neural tissue at day 0, in which neural cells were injected in hydrogel in the brain channel. A mixed medium of neural expansion medium and astrocyte medium was also added to the blood-side channel and the CSF-side channel at day 0. On day 3, a mixed medium of neural differential medium and astrocyte medium was added to the blood-side channel and the CSF-side channel. On day 5, vascular development began. Vascular cells in a mixed medium of endothelial cell medium and pericyte medium were injected into the blood-side channel, and a mixed medium of neural differential medium and astrocyte medium was added to the CSF-side channel with flow. On day 10, ischemia was induced by adding media without glucose to the blood-side channel and the CSF-side channel and incubating the microfluidic chips at 2% oxygen. On day 11, stem cell transplantation was performed by injecting stem cells in a mixed medium of endothelial cell medium and pericyte medium in the blood-side channel and adding a mixed medium of neural differential medium and astrocyte medium to the CSF-side channel. On day 18 the results were analyzed. This process is described in more detail in Table 1.

TABLE 1

| | Cell component | Culture condition | | | |
|---|---|---|---|---|---|
| | | blood-side channel | CSF-side channel | O₂ Conc. | Flow |
| Day 0 | Add NPC, astrocytes and microglia (8:4:1) with hydrogel into the ‘brain’ channel. | Add the mixture of NEM, AM and ACM (8:4:1, v/v/v) - serum free | Add the mixture of NEM, AM and ACM (8:4:1, v/v/v) | 20% | No |
| Day 3 Neuronal differentiation | | Add the mixture of NDM, AM and ACM (8:4:1, v/v/v) | Add the mixture of NDM, AM and ACM (8:4:1, v/v/v) | 20% | No |
| Day 5 BBB construction | Add pericytes and BMEC (9:1) into the ‘blood-side’ channel. | Add the mixture of ECM and PM (9:1, v/v) - 4.7 % (v/v) serum | Add the mixture of NDM, AM and ACM (8:4:1, v/v/v) | 20% | Yes |
| Day 10 Ischemia induction | | Add glucose/serum - free DMEM | Add glucose/serum - free DMEM | 2 % | No |
| Day 11 Reperfusion and stem cell transplantation | Add stem cells into the ‘blood- side’ channel. | Add the mixture of ECM and PM (9:1, v/v) | Add the mixture of NDM, AM, and ACM (8:4:1, v/v/v) | 20% | Yes |
| Day 18 | Lyse the whole tissue for analysis. | | | | |

NPC: Neural progenitor cell
NEM: Neural expansion medium
AM: Serum-free Astrocyte medium
ACM: Astrocyte- conditioned medium
NDM: Neural differentiation medium
ECM: Endothelial cell medium
PM: Pericyte medium

For the hydrogel matrix, a soluble form of basement membrane purified from Engelbreth-Holm-Swarm (EHS) tumor (Cultrex™, Trevigen) with major components of laminin, collagen IV, entactin, fibronectin, entactin, and heparan sulfate proteoglycans was used. The basement membrane derived from EHS tumor has an elastic modulus of around 0.5 kPa, within the range of the physiological stiffness of brain tissues, and supports the neuronal differentiation of NPC, as well as neuronal survival and functions. In previous chip designs, each microfluidic channel was separated by micro-poles to generate the surface tension necessary to confine the liquid hydrogel prepolymer within the designated channel. An endothelium was to be formed on the side wall of the hydrogel in the ‘brain’ channel, perpendicular to the x-y plane of the entire structure. However, it was discovered that these micro-poles interfered with the endothelial cells and kept them from forming a continuous and intact endothelium, causing physical defects. These defects could hinder the assessment of the actual efficacy of the stem cell therapeutics, because the defects could serve as an artificially easy shortcut for the stem cells or bioactive substances in the bloodstream to reach the ischemically damaged brain.

Thus, a new chip design without any micro-poles but still with the real-time monitoring capacity in the same microscopic focal planes was developed. With the new design, the height of the middle ‘brain’ channel was lowered to generate the surface tension between the top and the bottom surfaces and to stably hold the liquid hydrogel prepolymer in the ‘brain’ channel. A well-defined boundary was formed between the ‘blood-side’ and ‘brain’ channels in the new chip. The reconstructed endothelium successfully prevented free diffusion of a fluorescent probe (FITC-dextran, 4 k Da) across itself. The probe size of 4k Da is useful to evaluate the BBB functionality because many pathogens, such as viruses and bacteria, are larger than 4k Da and the native BBB prevents these pathogens from entering the brain.

Single layer of the confocal microscopic images showed the continuous and physically intact endothelial barrier in contrast to the barrier produced with the previous chip design with micro-poles. Additionally, the new chip design is devoid of micrometer-scaled features, eliminating the need for the soft lithography process in chip production, and enables production with a 3D printer.

Before the endothelium formation, a small population of astrocytes were seen in the ‘blood-side’ channel after migrating from the ‘brain’ channel (0.9 ± 0.3 (s.d.) % of the totally incorporated astrocytes, n=3,). These migrated astrocytes, together with pericytes, supported BMEC to maintain the normal morphology of a smooth rounded shape throughout the ‘blood-side’ channel, like the morphology of BMEC when co-cultured with both astrocytes and pericytes in 2D culture. The astrocytes and pericytes in the ‘blood-side’ channel settled beneath the layer of the endothelial cells at the bottom, as the BMEC connected to each other, maturing to form an endothelium. It might be due to the angiogenic process in which endothelial cell-to-cell junctions strengthen the connection between the neighboring endothelial cells.

The endothelial tightness under different conditions, depending on the cell composition and the presence of flow, was examined by calculating the apparent permeability coefficients. In the presence of astrocytes and pericytes, the endothelium became significantly tighter to hinder the diffusion of the probe, FITC-dextran. Further significant reduction in the permeability was observed after the flow of culture media was introduced. This tightening of the endothelium in the presence of flow is in line with the reports of enhanced paracellular connectivity in BBB by proper mechanical stimuli. The resulting permeability coefficients of the BBB model were ~6×10⁻⁷ cm/s and ~ 8×10⁻⁸ cm/s for 4 k Da and 70 k Da FITC-dextran, respectively, comparable to those of other in vitro and in vivo BBB models previously reported. The reconstructed BBB also showed the expected size-selective permeability as in functional BBB; the smaller the probe size, the better the diffusion across the BBB.

Another standard measure to assess the BBB tightness is Trans-Endothelial Electrical Resistance (TEER). TEER measurement is a simple, label-free and non-invasive method to quantify the barrier integrity. There is a broad range of TEER values reported for microfluidic BBB models, from a few hundred to thousands of Ω•cm², while the permeability coefficients are within a relatively narrow range of around 1×10⁻⁶ cm/s for 4 k Da FITC-Dextran. This might be because the TEER values are largely dependent on the method of measurement and experimental procedures. Alternative currents (AC) are widely used for TEER measurement because direct currents (DC) can damage cells. And tetrapolar AC TEER measurement, using four electrodes, is more accurate than bipolar AC measurement as it is less influenced by the polarization impedance at the electrode-electrolyte interface. Due to the small surface area of the BBB in the chip, however, the resistance across the BBB was expected to reach several mega-ohms, beyond a measurable range of tetrapolar AC TEER meters commercially available. Thus, a bipolar DC measurement was used and the TEER value of the BBB in the chips was 370 ± 20 (s.d.) Ω•cm² under the flow. The TEER value measured was lower than those reported in some of the microfluidic BBB models, but showed meaningful differences between conditions.

Once the physical intactness of the endothelium was confirmed, the functional characteristics of the reconstructed endothelium as a bio-chemically intact barrier was examined. One of the important functions of the cerebral endothelium in vivo is to isolate the neural cells in the brain parenchyma from any pro-inflammatory substances in the bloodstream. To maintain the original phenotype of the cells in each channel of the chip, two different types of media: serum-containing endothelial media in the ‘blood-side’ channel and serum-free glial cell media in the ‘CSF-side’ channel were deployed. The reason for this setup is that the endothelial cells require serum to maintain their original phenotype in vitro, whereas the glia cells show pro-inflammatory behaviors in the serum-containing culture medium. Serum, extracted from the whole blood, is an undefined mixture of proteins, hormones, minerals, growth factors, and lipids. The reconstructed BBB thus needs to prevent the entry of any pro-inflammatory substances from the serum in the ‘blood-side’ channel. In the samples without BBB, the microglia, a resident immune cell type in the brain, showed pro-inflammatory behaviors as expected because they were directly exposed to the serum. In contrast, in the samples with the reconstructed BBB, the microglia did not show such pro-inflammatory behaviors, confirming the BBB in the chip as a bio-chemically intact barrier, similar to the native BBB.

To be a clinically relevant platform for stem cell therapy, the BBB on the chip should also exhibit distinct responses based on the traits of the invading cells. The neurorestorative efficacy of each stem cell type may depend on their capacity to infiltrate across the tight BBB and reach the lesion site, and yet little is known about the native BBB responses to the candidate types of stem cells in therapy. Therefore, as a valid measure to show the cell-selective responsivity of the BBB, the well-established metastatic behaviors of the two human breast cancer cell lines, MB-231 and its brain metastatic derivative population, MB-231Br were used. MB-231Br infiltrates specifically across the BBB and exhibits much stronger metastatic tendency than MB-231 in an animal model. The reconstructed BBB showed the expected cell-specific responses to these two types of invading cancer cells, confirming the in vivo-like functionality and verifying its sensitivity to the traits of the invading cells.

### Establishing the Ischemia

After confirming the formation of a functional BBB in the chip, an ischemic condition was established. There are two major zones of ischemic injury: the core infarct zone and the ischemic penumbra, also called the peri-infarct rim. The core infarct zone is characterized by no blood supply and severe necrosis of neural cells, and is considered irreversibly injured. In contrast, the ischemic penumbra, the rim surrounding the irreversibly damaged core, has just enough blood supply for the cells to survive but not enough to communicate and function properly. This peri-infarct rim has been considered as a therapeutic target for post-stroke recovery. Thus, it was targeted to establish an ischemic condition recapitulating this peri-infarct zone, sufficiently damaging the cells and yet minimizing cell death.

The optimized ischemic condition was 2% O₂ with depletion of serum and glucose for 24 hours, in the absence of flow. This ischemic condition sustained the cell viability while inducing detectable cytotoxicity measured by the amount of the extracellular lactate dehydrogenase (LDH) released through the damaged cell membranes. It was also observed that hypoxia-inducible factor-1α (HIF-1α), usually found in the cytoplasm of the cells under normoxic condition, translocated to the nucleus, as observed in the ischemic brain in vivo. According to the gene expression alteration pattern, the ischemic insult upregulated the genes in both the apoptotic and the antiapoptotic signaling cascades, just as reported in animal ischemic stroke model. Oxidation-reduction reaction was also upregulated, implying the cells protected themselves against the elevated intracellular levels of reactive oxygen species in ischemia. The upregulation of the neurotrophic and angiogenic factors suggest the attempts of the ischemically damaged cells to repair and remodel themselves. The cells also exhibited typical neuroinflammatory responses against ischemic stroke as shown in the upregulated gene expressions of pro-inflammatory cytokines and Integrins. The downregulated expression of extracellular matrix proteins, together with the enhanced activities of matrix metalloproteinases and the decreased interaction between the cells and ECM imply that the ischemic insult led to the impairment of tissue integrity as well as the subsequent tissue remodeling process. Overall, these gene expression patterns collectively indicate that the ischemic condition successfully induced inflammation and deterioration in tissue integrity as expected and also accompanied endogenous neuroprotection and tissue remodeling, as reported in many other in vivo stroke models.

### Verifying the NVU Behaviors

To verify the functionality of the reconstructed NVU, individual cell behaviors at various levels both under healthy and ischemic conditions were examined. At gene level, the expression alteration of the genes associated with a series of post-stroke pathological conditions were measured and categorized based on their functional characteristics. As most of the genes are not cell-specific and involved in multiple cellular processes, this grouping is solely for the purpose of outlining the overall pattern of the responses across the cell population in the experiments.

Neurons are the primary component of the central nervous system and play critical roles in neurological functions. Considering the short lifespan and limited expansion capacity of the primary human neurons in vitro, human iPSC (induced pluripotent stem cell)-derived neural progenitor cells (NPC) were used in the stroke model and the culture conditions of the chip was optimized for their neuronal differentiation. The differentiated NPC exhibited the neuronal morphology of a cell body and branches of axons and dendrites, and expressed mature neuron markers such as microtubule-associated protein 2 (MAP-2) and Synapsin I and II (SYN), a family of proteins regulating neurotransmitter release at synapses. They also maintained proximity with the astrocytes. Under ischemic condition, they showed dendritic beading or fragmentation, a typical morphology of degenerating neurons, compared to the smooth and clear dendritic morphology observed in normoxia. They were also stained by a neuronal degeneration marker Fluoro-Jade stain, consistent with the reports from in vivo ischemic stroke models.

The gene expression alteration by the ischemia shows that the endogenous repair led to the upregulation of the gene groups involved in Neurite formation and Synaptogenesis, but it was accompanied by downregulation of genes related to Synaptic plasticity. The excessive stimulation of an excitatory neurotransmitter, glutamate, and at the same time the decreased activity of an inhibitory neurotransmitter, Gamma-Aminobutyric acid (GABA) (ABAT and GABRB1 are encoded in an enzyme for GABA catabolism and in one of the GABA receptors, respectively) were also observed. These expression patterns imply the disrupted balance between neuronal excitation and inhibition in the ischemic condition, potentially leading to the excitotoxicity typically observed in ischemic stroke.

How the ischemic condition was reflected in the cytosolic calcium (Ca²⁺) oscillation pattern in the differentiated NPC was also examined. The cytosolic Ca²⁺ imaging provides an indirect but accurate measure of the action potential generation in individual neurons, and represents various neuronal functions ranging from synaptic activity to cell-cell communication, adhesion, neurodegeneration and apoptosis. The cytosolic Ca²⁺ images show that the differentiated NPC exhibited the typical four patterns of cytosolic Ca²⁺ signals: oscillatory (repeated brief increase in free Ca²⁺), transient (brief elevation due to Ca²⁺ influx through membrane calcium channels), sustained (sustained increase in Ca²⁺ level by both external and internal stores), or unnoticeable signals. The ischemic insult decreased the ratio of cells showing the unnoticeable Ca²⁺ signals, while increasing the ratio of cells showing both transient and sustained signals. The accumulated Ca²⁺ level in the cytoplasm is thought to lead to neuronal death in animal stroke models. Analysis on the oscillatory signal alteration by ischemia reveals insignificant changes in the amplitude but significant increase in frequency of the oscillation, indicating the increased Ca²⁺ influx into the cells. The excessive influx of Ca²⁺, together with the disrupted balance between neuronal excitation and inhibition, show excitotoxic neurodegeneration in the ischemic samples.

Brain Microvascular Endothelial Cells (BMEC) are the primary cellular component of the cerebral vasculature, BBB. Human primary BMEC were used throughout. BMEC have a high mitochondrial density, lack of fenestrations, low pinocytic activity, and high density of adherent and tight junctions compared to the endothelial cells found in other tissues. The tight junctions determine the paracellular tightness of the endothelial cells and the permeability across BBB. Zonula occludens-1 (ZO-1) is a dominant junctional adaptor protein, regulating other junctional components, cell-cell tension, angiogenesis, and BBB formation The flow through ‘blood-side’ channel increased the expression of ZO-1 and caused the shape of the cell body to elongate along the direction of the simulated bloodstream. The upregulated ZO-1 expression in the samples with flow led to the upregulated expression of other junctional proteins, VE-cadherin and Claudin-5. The ZO-1 expression of the ischemic samples significantly decreased compared to the samples in normoxia with the flow, but was statistically comparable to the normoxic samples without the flow. Importantly, the expression of ZO-1, mainly localized on the cell membrane under normoxia conditions, spread throughout the cell body under ischemic conditions. This dispersed spatial distribution of ZO-1 in the ischemic samples led to the increased permeability of fluorescence probe (4k Da FITC-dextran), representing the reduced paracellular tightness under ischemia. These results suggest that the paracellular tightness among the endothelial cells is affected more significantly by the extent of tight junction localization on the cell membrane, rather than the overall level of their expression. The BMEC in the ischemic samples significantly increased the expression of the vascular endothelial growth factor (VEGF), one of the angiogenic factors, suggesting the post-stroke vascular reorganization took place, as observed in an animal stroke model. At the gene level, the ischemic insult decreased the endothelial paracellular connectivity, but upregulated the genes involved in Vasoconstriction and Adhesion molecules for recruiting immune cells, as observed in animal stroke models.

BMEC behaviors have been well documented in various experimental conditions. In a mono-culture of human BMEC, the shear stress induced by the flow did not significantly affect the expression of the tight junction proteins or their morphology. On the other hand, the flow condition in a mono-culture of bovine BMEC led to the upregulation of tight junction proteins and the morphological alignment along the flow direction. In another in vitro work, rat BMEC required the appropriate interactions with both the astrocytes and the pericytes to show their original pattern of tight junction localization around the cell membrane, as was observed in this model. Taken together, these results suggest that in order for human BMEC to exhibit in vivo-like behaviors, they need some of the key components of the original BBB microenvironment: the mechanical stimuli by the blood flow and the heterocellular network in NVU. This stroke model provides both of these microenvironment features, allowing for in vivo-like behaviors of human BMEC.

Pericytes are mural cells of the microvasculature, and regulate BBB permeability, angiogenesis, clearance, cerebral blood flow, neuroinflammation and stem cell activity. Human primary brain vascular pericytes were used. The pericytes in the chip expressed platelet derived growth factor receptor beta (PDGFRβ), one of the pericyte specific makers, and positioned themselves between the mature endothelium and the side wall of the ‘brain’ channel. The pericytes were activated in response to the ischemic injury, contributing to vascular inflammation. The interaction of pericytes with endothelial cells, which is crucial for the vascular stability under normal condition, was downregulated.

Astrocytes are the dominant glial cell type in the brain and play many mediating roles in the heterocellular interactions in NVU. Human primary astrocytes were used throughout. One of their roles is to sense neuronal metabolic activities and coordinate vasodilation and vasoconstriction to match the blood flow accordingly. Astrocytes carry out these intermediary roles through direct contact-based interactions with the endothelial cells. Oxygen and nutrients were provided only through the ‘blood-side’ and ‘CSF-side’ channels so that the astrocytes in the ‘brain’ channel would have to migrate and extend their endfoot toward the formed endothelial layer at the boundary to access the nutrients, thus forming physical contact with it. This physical contact was indirectly confirmed through the immunofluorescence staining of water channel proteins encoded by Aquaporin-4 (AQP4), the most abundant water channels in the brain. The water channels in astrocytes are localized around astrocytic endfoot in direct contact with the blood vessel under normal conditions. This polarized location reflects their mediating role in gaseous exchange including O₂, CO₂, and NO. In inflammatory conditions like ischemia, the immunoreactivity of AQP4 in astrocytes bleeds away from endfoot, implying the disruption of the mediating role of AQP4. In addition, the astrocytes in the ischemic samples showed the reactive astrogliosis, characterized by abnormal hypertrophy, massive proliferation, and upregulated Glial Fibrillary Acidic Protein (GFAP) expression levels. Astrocytes failed to show these behaviors in the traditional 2D culture conditions. The gene expression pattern reveals a heterogeneous population of astrocytes mixed with both A1 (inflammation-induced) and A2 (ischemia-induced) phenotypes, as reported in in vivo stroke models. The activated astrocytes in turn decreased their trophic support for the neurons in the ischemic stroke model, consistent with the reports from other stroke models.

Microglia are the resident macrophages and the only immune cell type in the brain. Due to the issues of reliable batch-to-batch reproducibility with human primary microglia, a transformed human microglial cell line (HMC3, ATCC) was used. Microglia in the brain show immediate pro-inflammatory responses to any injury or infection. Once activated, their pro-inflammatory morphology changes are signified by the retraction and thickening of the processes, and the hypertrophy of the cell body, which was successfully reproduced in the ischemic samples. They also promptly secrete interleukin-1β (IL-1β), one of the pro-inflammatory (M1) phenotype markers, within a few hours of the inflammation onset. This upregulation of IL-1β, though, is only temporary and not sustained. On the other hand, the expression of the cluster of differentiation 68 (CD68) and ionized calcium-binding adapter molecule 1 (IBA-1) is upregulated during M1 phase and persists throughout the anti-inflammatory (M2) phase thereafter. These in vivo-like temporal patterns of the IL-1β and CD68 immunoreactivity were observed in the stroke model. In contrast, the traditional 2D culture conditions failed to induce these behavior changes in microglia. The gene expression pattern indicated that the ischemic onset led to the upregulation of both pro- (M1 phenotype) and anti-inflammatory (M2a and M2b phenotypes) microglial markers, as observed in in vivo studies. Both M2a and M2b are involved in phagocytosis and produce anti-inflammatory cytokines, although their activation signal pathways are distinct from each other. In contrast, M2c phenotype, usually regarded as a marker for the deactivating stage, barely appeared in the 24-hour time frame after the ischemic onset in the stroke model, consistent with the report that M2c macrophages appeared only after the downregulation of the inflammation. Many other Immune receptors and Chemoattractants also showed upregulated expression level. The genes engaging both the innate and the adaptive immune responses were generally upregulated in the ischemic condition, as previously reported. The gene expression of the purinergic receptors, involved in both immune cell regulation and neurogenesis, appeared rather inconsistent, although it was also clear that the overall immune responses were not well-regulated right after ischemia. These gene expression alterations indicate that the ischemic insult triggered a broad spectrum of immune responses, from exacerbating the ischemic injury to helping repair, as observed in other ischemic stroke models.

### Characterizing the Neurorestorative Potential of Stem Cells

Substantial amount of studies has supported the neurorestorative potential of stem cells for stroke treatment, but there have also been a few reports contradicting some of these observations. This could be partially because the experiments were all conducted under different conditions and/or focusing on different aspects of the complicated recovery processes. Being an in vitro system, the present stroke model allows for the identical experimental conditions across large number of samples and over repetitions. It thus serves as an effective platform to systematically examine the neurorestorative capacity of clinically relevant stem cells. The stem cells examined in this stroke model include human induced pluripotent stem cell derived neural progenitor cells (hNPC), human embryonic stem cell derived neural stem cells (hNSC), human hematopoietic stem cells (hHSC), bone marrow derived mesenchymal stromal/stem cells (hBMSC), adipose derived mesenchymal stromal/stem cells (hAMSC), and endothelial cell progenitor cells (hEPC). The effect of reperfusion treatment only, without stem cells, was examined by re-introducing oxygen and glucose after ischemic insult.

The neuro-restoration after ischemic stroke entails an expansive series of processes from neural cell regeneration and immune suppression, to restoration of vascular structures, and to recovery of heterocellular interactions in NVU. 123 relevant genes involved in each of these aspects were selected based on the Human Neurogenesis PCR array (Qiagen) as well as experimental data on the ischemic responses in the chip. The overall gene expression of the chosen set was generally upregulated by the incorporation of all types of stem cells as well as the reperfusion only. When the genes with over 4-fold changes in expression were considered, hNPC and hNSC were mostly associated with strongly upregulated genes, while the opposite was true for hBMSC. hEPC turned up almost equal numbers of strongly up- and downregulated genes.

The stem cell incorporation as well as reperfusion generally invoked positive influence on the generation of the cells in the nervous system (Neurogenesis), although the extent of this influence varied across stem cell types. In addition, all groups enhanced the expression of genes involved in Neuron migration, Neuron differentiation, Neuron fate commitment, Axonogenesis, and Gliogenesis, although hBMSC also showed inhibiting influence on Neuron differentiation and Neuron fate commitment. Notably, the reperfusion upregulated the expression of all the genes involved in neuronal migration, even though the extent was weak. As for the synapse responses, similar pattern is revealed in Synapse organization and Regulation of synapse plasticity, with dominantly positive influence from all experimental group, except for the fact that hEPC and reperfusion exhibited equally strong enhancing and inhibiting effects.

In the post-stroke recovery process, it is also important to suppress the inflammation initiated by the ischemia. The hNPC and hNSC most strongly upregulated inflammation-related genes. While hAMSC also slightly upregulated, hAMSC, hBMSC, hHSC and perfusion slightly suppressed the genes in Inflammation response group. To examine more specific inflammatory responses, the expression of glial phenotype markers was measured and the influence of the translated stem cells on their inflammatory behaviors was examined.

As for the effects on BMEC, hNPC, hNSC and hEPC upregulated the expression of the tight junction protein 1 (TJP1), but the expression of the Claudin 5 (CLDN5) was downregulated in all groups, despite the fact that TJP1 and CLDN5 interact closely to form BBB. The expression of PECAM1, one of the endothelial adhesion molecules responsible for immune cell recruiting after brain injury, was all effectively downregulated. As for the effects on pericytes, only hAMSC suppressed the expression of a reactive pericyte marker, CSPG4. The expression of CD248, involved in the role of pericytes in mediating angiogenesis, was upregulated by hNSC and hAMSC only, and generally suppressed by the rest. Regarding the influence on the microglial activities, all groups failed to suppress the expression of a microglial reactive marker, CD68, usually upregulated throughout the whole inflammatory phase. In contrast, CD 86, a proinflammatory M1 phenotype marker was successfully suppressed by all groups, except for hAMSC and hHSC. hHSC enhanced the expression of CD206, an anti-inflammatory M2a phenotype marker, while hEPC and hBMSC promoted the expression of CD32a, an anti-inflammatory M2b phenotype marker. hBMSC, hAMSC and reperfusion upregulated the expression of CD163, a microglia-deactivating phenotype marker. As for the effects on astrocytes, there was no group suppressing the expression of VIM, a pan reactive astrocyte marker. hNPC was the only one that successfully suppressed the expression of C3, an A1(inflammation) reactive astrocyte marker. In all groups, the expression of CD109, a A2 (ischemia) reactive astrocyte marker, was downregulated. Taken together, the expression pattern of these astrocyte reactive markers suggests that 7 days after the ischemic insult there was little influence from the ischemia itself, and yet the astrocytes still retained their inflammatory behaviors. The expression of IFITM3, involved in a neurotrophic support of astrocytes, was upregulated in all groups, while the expression of FABP7, another gene with similar function, was upregulated only by hNPC, hEPC, and hBMSC. The complexity in the overall gene expression pattern suggests that all six types of stem cells as well as reperfusion have their own pathways to suppress the neuroinflammation induced by the ischemia.

When the gene expression pattern was hierarchically clustered, hNPC and hNSC, with more restricted fate commitment to neural cells, stood out as a group separate from the rest. The differential expression analysis between the two groups (group with neural differential capacity (NDC) vs. group without NDC) identified 27 genes. The GO enrichment analysis on the identified genes was performed based on the STRING database and found that the stem cells with NDC were beneficial in Neurogenesis and other closely related GO terms. The stem cells with NDC also had positive effects on regulating the signal cascade of Mitogen-Activated Protein Kinase (MAPK), an important regulator of ischemic and hemorrhagic cerebral vascular disease.

To better distinguish the neurorestorative characteristics of each type of the stem cells, the GO enrichment analysis was performed focusing on the genes with over four-fold expression changes after stem cell incorporation to identify the dominant therapeutic pathways for each stem cell type. hNPC showed the greatest potential for Neurogenesis, especially regarding the Generation of neurons, though it had stimulating effects on other restorative functions as well. The influence hNSC is more evenly spread out across diverse aspects, such as forming and maturing tissue structures and developing multicellular organism. hNSC also showed strong capacity in developing blood vessel, a fundamental environment for restoring NVU function, as well as promoting the movement of cells, an important feature for reorganizing the ischemically damaged structure. Other relative advantages of hNSC included enhanced adaptation to environmental changes, and better regulation of immune. Notably, hNSC strongly suppressed the acute inflammatory responses. hNSC was also least associated with the Pathways in cancer based on the Kyoto Encyclopedia of Genes and Genomes (KEGG) database. Across the whole range of the GO terms, hEPC exhibited the tendency of simultaneous promotion and inhibition, while hBMSC consistently inhibited most of them.

Compared to the top three influential stem cell types (hNPC, hNSC, and hEPC), the rest of the experimental groups (hBMSC, hAMSC, hHSC, and reperfusion therapy) induced relatively smaller changes in gene expression. The GO enrichment analysis on the genes with over two-fold expression changes was also performed for these groups. Unlike the distinct characteristics emerged from the previous analysis for the three most influential stem cell types, these groups exhibited relatively inconsistent influences over the GO terms grouped together for related functions. A few consistent trends were Neurogenesis promotion by hHSC and Vasculature development inhibition by hAMSC and reperfusion.

The synaptic activities were further examined as an important and reliable parameter to estimate the extent of the post-stroke recovery. Based on the GO enrichment analysis on the genes with at least 4-fold or 2-fold expression changes, the significance of the GO terms related with the synapse and neurotransmitter was compared, and how much each stem cell type promoted the synaptic activities was examined. This analysis also revealed a streak of strong positive effects of hNSC on many aspects of the neurorestoration at synapse level: Synapse Organization and Synaptic Transmission, as well as the regulation of the transmission pathways of the relevant neurotransmitters such as Glutamate, Acetylcholine, and GABA. Given the molecular and functional complexity of the synapses and the importance of their coordination in neurological functions, hNSC stand out as the highly promising therapeutic agent among all stem cells evaluated.

### Tracking the Transplanted Stem Cells

Although the exact mechanism underlying the neurorestorative effects of the transplanted stem cells for stroke is still unknown, there are accumulating evidences that the therapeutic effects of stem cell therapies are mediated by indirect mechanisms, such as releasing trophic factors and immune regulatory cytokines, promoting endogenous stem cell migration, and enhancing endogenous neural plasticity and function recovery. However, albeit rarely, there also have been reports that the transplanted stem cells directly replace the host cells, reconstituting the damaged neural circuitry. The primary factor attracting the stem cells toward the infarcted brain parenchyma seems to be the inflammatory responses of NVU, such as the upregulation of cytokines, CAM, and MMP, which is observed in this NVU model as well.

With this stroke model, it was possible to track the transplanted stem cells and assess the extent of the direct cell replacement. Each of the major indicators of the cell replacement were examined: the extent of adhesion to the BBB, the number of surviving cells, the extent of infiltration into the ‘brain’ channel and differentiation into various neural cell types in NVU. First, GFP-expressing stem cells were prepared using lentiviral factors. The number of stem cells initially adhering on the BBB was less than 5 % of the total cell number in the chip in most cases. After seven days of stem cell injection, the cell viability counts for those attached to the BBB were in general either decreased (hNPC, hNSC, and hHSC) or only slightly increased (hBMSC and hAMSC). hEPC, in contrast, vigorously proliferated and infiltrated into the ‘brain’ channel. At the same time point, seven days after transplantation, all of these transplanted stem cells barely expressed their stem cell markers that they originally expressed in 2D cultures. This does not mean they successfully completed differentiation by that time because only very limited number of the cells were mature enough to express the markers of their predicted lineages. Only hNPC and hNSC showed detectable neural differentiation and even those were less than 0.01% of the total cell number in the chip. The extremely limited stem cell differentiation suggests that the direct cell replacement is not a major mechanism underlying the neurorestorative effects of stem cell therapy, adding to the recent growing evidences against it.

### DISCUSSION

The data revealed three key aspects of NVU microenvironments required for in vivo-like behaviors of the constituent cells: the formation of intact BBB, the heterocellular network, and the proper mechanical stimuli by blood flow. The brain-like microenvironment ensures the cells in this model to retain their native behaviors and to show clinically relevant responses to an ischemic insult. This model served as an efficient screening platform to examine the neurorestorative potential of the stem cells used in pre-clinical trials. How each type of stem cells influenced the gene activities during the complicated disease progression and recovery processes was systematically analyzed. This stroke model was also used to track the stem cell behaviors transplanted in the ischemically damaged NVU.

This microfluidic chip design well suited the need of establishing a functional BBB and at the same time enabled the real-time monitoring of the transplanted stem cells moving across the BBB. Similar chip designs have been proposed to build a functional BBB: positioning cells side-by-side by using micro-poles (AIM Biotech) or a flow-guiding structure (PhaseGuide ™ technology, Mimetas). The design of such a chip is useful for observing the behavior of drugs or cells passing through the BBB in a 3D environment.

What differentiates this design from prior designs, such as those using phase guides or porous membranes, is the absence of physical structures between two neighboring channels, which allows for the cellular interactions free from any potential interference due to physical structures.

The stroke model successfully delineated the neurorestorative behaviors of each candidate stem cell type for stroke treatment. The benefits of hNPC and hNSC, the stem cells with the capacity to differentiate into neural cells, consistently stood out in many aspects related with the post-stroke recovery processes. The iPSC-derived hNPC (Millipore, Cat. No.: SCC035) were tested by the manufacturer to ensure more than 80% of their progeny to differentiate into neuronal cells. The hNSC used in this work were initially isolated from fetal cortical brain tissue at 13.5 weeks gestation (M031 clone) and classified as neural stem cells due to their ability to self-renew and produce progeny cells differentiating into neural cells. Based on the GO analysis, hNPC showed the strongest capacity in generating neurons and hNSC exhibited compelling positive effects on the overall structural and functional integrity in NVU. Notably, the recovery of NVU functionality, such as gliogenesis, blood vessel development, and immune system process, was also linked with the enhanced synaptic activities, both mediated by hNSC. Given the importance of the synaptic activities in rewiring neuronal network and neurological functions, this result suggests that restoring the overall NVU functionality may be more critical for stroke treatment than replenishing neurons themselves. It is important to take into account the limitations of this approach as well when interpreting these results. First, the efficacy evaluation focused only on the gene level, as represented by GO functional analysis, and did not cover the entire range of interactions across different levels associated with post-stroke recovery. The transcriptomics was also performed on the whole cell population, which has limitations in showing cell- or tissue-specific changes. And there is always the risk of overinterpretation of the GO analysis results. Second, the contribution from the peripheral immune cells crossing BBB was not addressed in this model and could also play important roles in the post-ischemic inflammation. Because the incorporated neurons differentiated from neural progenitor cells rather than mature neurons, the chip could contain subsets of neurons with heterogeneous maturity. Third, since the flow in the chip was bidirectional, generated by a rocking shaker, the endothelial cells would activate different signal pathways of mechanotransduction compared to the unidirectional blood flow in vivo.

The results from tracking the transplanted stem suggest that the therapeutic effects of the stem cells arise mainly through the indirect mechanism of supporting the endogenous recovery, rather than direct cell replacement. At the time of gene expression alteration analysis, the number of stem cells left in the samples was mostly less than 1% of the whole cell population. The presence of such a small population itself could not possibly be the major driving force to induce the observed magnitude-fold changes in the gene expression for the whole cell population. This implies that the presence of the remaining stem cells themselves have had a minor influence on efficacy evaluation. Similar observations were reported in both animal models and clinics that the transplanted stem cells barely reached the ischemic region, but still induced significant therapeutic effects. Based on these observations and implications, the pre-clinical evaluation of the candidate stem cells for cell therapy would be more effective and relevant if focusing on their capacity of restoring the damaged NVU both structurally and functionally, rather than tracing the fate of the transplanted stem cells themselves in vivo.

Many of the previous studies have presented conflicting results, not only on the neurorestorative potential of each stem cell type in varying conditions, but also on the mechanism by which stem cells exert their therapeutic effects. The possible reason for these controversies could be the fact that the efficacy evaluation was focused only on a few aspects, lacking comprehensive analysis on the overall recovery process. Another reason could be the comorbidities often accompanying stroke, such as hypertension, high cholesterol, and diabetes, that complicate the disease progression and treatment. As such, stem cell therapy would be most effective with personalized approach based on the comprehensive health condition of individual patients. In vitro stroke models, like the one described herein, would serve as an ideal platform to develop personalized stem cell therapies, by utilizing patient-derived cells and simulating the unique pathophysiological condition of individual patients. The personalized stroke model could in turn serve as an efficient test-bed to screen many different candidate stem cells and identify the optimal stem cell regimen for the given patient. Multiomics approach, presented in some of the recent studies, could further expand the understanding of the post-stroke neurorestoration process and the in vitro stroke model is readily applicable for that purpose as well.

Taken together, the approach successfully recapitulated the NVU behaviors in the normal and ischemic conditions in vitro and enabled efficient and systematic evaluation of the stem cell therapy, overcoming the limitations of both the animal models and the currently available in vitro models. These findings, especially the characterization of the neurorestorative potential of various stem cells, can steer the direction of the advanced stem cell therapeutics in research as well as in clinics. The experimental platform presented is also immediately applicable to a wide range of other diseases associated with the vasculature, opening up new possibilities in the field of precision medicine.

### METHODS

### Cell Culture

### NVU Constituent Cells

Human primary astrocytes (ScienCell, Cat. No.: 1800) were cultured on T75 pre-coated flask with 2% poly-L-lysine solution (Sigma) in an astrocyte medium (AM) (ScienCell, Cat. No.: 1801). Transformed human microglial cell line (HMC3, ATCC, Cat. No.: CRL-3304) was maintained in Eagle’s Minimum Essential Medium (EMEM, ATCC) containing 10% fetal bovine serum (FBS) and 1% penicillin/streptomycin. Human induced pluripotent stem cell (hiPSC)-derived neural progenitor cells (hNPCs) (Millipore, Cat. No.: SCC035) were maintained on T75 pre-coated flask with 1% Matrigel (BD Matrigel Matrix High Concentration) in NEM. Human primary brain microvascular endothelial cells (BMEC) (ScienCell, Cat. No.: 1000) were cultured on T75 pre-coated flask with 2% collagen solution (Sigma) in an endothelial cell medium (ECM) (ScienCell, Cat. No.: 1001). Human brain vascular pericytes (ScienCell, Cat. No.: 1200) were grown on T75 pre-coated flask with 2% poly-L-lysine solution (Sigma) in a pericyte medium (PM) (ScienCell, Cat. No.: 1201). T75 Flasks coated with Matrigel and those coated with poly-L-lysine solution were prepared through incubation at 37° C. for 1 h and overnight, respectively. T75 flasks coated with collagen were prepared through incubation at 4° C. overnight.

### Stem Cells

Human endothelial progenitor cells (hEPC) were purchased from Celprogen (San Pedro, Cat. No.: 37089-01) and were expanded on T75 flasks pre-coated with the extracellular matrix for hEPC expansion (Celprogen, Cat. No.: E36053-05-T75) in complete hEPC growth medium (Celprogen, Cat. No.: M36053-05ES). Human bone marrow-derived mesenchymal stem cells (hBMSC, Gibco, Cat. No.: A15652) and human adipose-derived mesenchymal stem cells (hAMSC, Gibco, Cat. No.: PCS-500-011) were maintained in a mesenchymal stem cell medium (MSCM) (ScienCell, Cat. No.: 7501). Human neural stem cells (hNSC, NR1), initially isolated from fetal cortical brain tissue at 13.5 weeks gestation (M031 clone) and derived from the embryonic stem cell line H9, were cultured in the same condition as the hiPSC-derived NSC. Human hematopoietic stem cells (hHSC) were purchased from ATCC (Cat. No.: PCS-800-012) and used directly for experiments without subculturing. Medium was changed every 2-3 days. Cells were passaged when the confluency reached approximately 80%. 0.25% trypsin-EDTA was used to passage transformed microglia, hBMSC, and hAMSC. 0.05% trypsin-EDTA was used to split astrocytes, BMEC, pericyte, hEPC, and hNSC. hNPC was passaged using StemPro™ Accutase™ Cell Dissociation Reagent (Gibco, Cat. No.: A1110501).

### Microfluidic Chip Design and Fabrication

The master mold of microfluidic chips was fabricated using a stereolithography 3D printer. The printed molds were extensively washed with 99% isopropyl alcohol to remove unreacted monomers and curing agents, and incubated on a hotplate at 50° C. in a UV light chamber (wavelength: 365 nm and 405 nm, output: 48 W) overnight. This washing process was repeated for at least 3 days before used for chip production. The surface of the molds then was spray-coated with silicone mold release (CRC, cat. No.: 03300) and PDMS (Sylgard 182, Dow Coming) was poured on it. After heat curing at 65° C. for approximately 5 h, the solidified PDMS replica was peeled off from the mold. Holes (1.5 mm in diameter) were made at both ends of each channel in the PDMS replica using a biopsy punch. The PDMS replica was then bonded to precleaned microscope glass slides (Fisher Scientific) through plasma treatment (Harrick Plasma, Cat. No.: PDC-32G). Microfluidic chips were UV-treated overnight for sterilization before cell seeding.

### Reconstruction of a Functional NVU Chip

### Reconstruction of Brain Tissue

To construct functional brain tissue on the microfluidic chips, human iPSC-derived NPCs, astrocytes, and microglia were embedded in a basement membrane extract (BME) hydrogel (Cultrex™ reduced growth factor basement membrane matrix type R1, Trevigen, Cat. No.: 3433-001-R1) and then injected into the ‘brain’ channel of the chips. hNPCs were suspended in a neural expansion medium (NEM, Millipore, Cat. No.: SCM004) supplemented with 2 mM glutamine and 0.02 µg/mL FGF-2. To obtain astrocytes and microglia in their resting state, they were sustained in AM without serum and astrocyte-conditioned medium (ACM, ScienCell, Cat. No.: 1811), respectively, for one day before the injection. The density of the suspension for each cell type was ~ 8 × 10⁶ cells/mL. The cell mixture was prepared by mixing hNPCs, astrocytes and microglia at the ratio of 8:4:1 (n/n/n) and then with BME Type R1 hydrogel prepolymer (gel: cell = 4: 1 (v/v)). According to the vendor, more than 80% of the hiPSC-derived NPC commit to mature neurons, making the final cell ratio for neurons, astrocytes and microglia fall in the range of 5-6: 4-5: 1 (n:n:n), similar to the naïve brain. The gel-cell mixture was injected into the ‘brain’ channel of a chip placed on a cold pack. The total number of the incorporated neural cells in the ‘brain’ channel was around 4 × 10⁴. After injection, chips were transferred to rectangular 4-well cell culture plates (Thermo Scientific, Cat. No.: 267061) and incubated at 37° C. in a cell culture incubator for 30 minutes for gelation. After gelation, the serum-free mixed medium of NEM, serum-free AM and ACM (8:4:1, v/v/v), (referred to as NEM/AM), was injected into both the ‘blood-side’ and the ‘CSF-side’ channels and then changed every day. From day 3 after the injection, NEM was replaced with a neural differentiation medium (NDM) (Millipore, Cat. No.: SCM111), referred to as (NDM/AM) in. The culture medium was changed every other day for the next 2 days until BBB reconstruction.

### BBB Reconstruction

BMEC and human pericytes were suspended in ECM and PM respectively at the density of ~ 1 × 10⁶ cells/mL. BMEC and pericytes were mixed at 9:1 (n/n) ratio based on literature and 10 µL of the cell suspension was injected into the ‘blood-side’ channel of a chip after the neural cells were co-cultured for 4 days in the ‘brain’ channel. The total cell number in the ‘blood-side’ channel was around 1 × 10⁴. The chip was slightly tilted for BMEC and pericytes to adhere to the side wall of the hydrogel in the ‘brain’ channel and incubated it for 3 hours. Then the old medium was removed and injected fresh mixed medium (ECM: PM = 9:1 (v/v), final serum content of 4.7% (v/v), referred to as ECM/PM, into the ‘blood-side’ channel to remove any unattached cells and debris. The mixed medium of ECM and PM in the ‘blood-side’ channel and the mixed medium of NDM, AM and ACM in the ‘CSF-side’ channel were changed every other day. Chips were cultured for 3 more days for BBB formation. In the experiments to track pericytes, BMECs and pericytes were pre-stained with DiD and DiO cell-labelling solution (Vybrant ™, Invitrogen), respectively, whole their nuclei were counterstained with NucBlue™ Live ReadyProbes™ Reagent (Thermo Fisher Scientific).

### Shear Stress on the BBB

To apply the shear stress of flow in the physiological range as in the brain microvasculature (0.01 - 10 dyne/cm²), a pulsatile bidirectional flow was generated by placing the samples on a rocking see-saw shaker (Mimetas, OrganoFlow^{®} L,). The design parameters of model were modulated based on the equation below:

$$τ = \frac{6 ⋅ Q ⋅ μ}{b ⋅ {h}^{2}} ,$$

where τ = shear stress (dyne/cm²), Q = flow rate (cc/s), µ = viscosity of culture medium, b = channel width, and h = channel height. Based on the Poiseuille’s law,

$$Q = \frac{Δ P ⋅ π ⋅ {D}^{4}}{128 ⋅ μ ⋅ L}$$

$$D = \frac{2 ⋅ h ⋅ b}{h + b}$$

(hydraulic diameter of a rectangular channel), where ΔP = ρ · L · sin θ (pressure difference between the inlet and outlet), θ = tilt angle of a shaker, L = channel length, ρ = liquid density. The mean shear stress during a given time period is proportional to the following parameters:

$$τ ∝ \frac{{h}^{2} ⋅ {b}^{3}}{{\left(h + b\right)}^{4}} ⋅ sin θ$$

In previous work with the experimental setup with h = 220 µm, b = 400 µm, θ = 7 ° (OrganoPlate, Mimetas), and the tilting frequency of 16 minutes, the maximum shear stress was estimated as 1.7 dyne/cm² based on a numerical model simulated in Python software. The present setup (h = 400 µm, b = 1 mm, θ = 4 °, and the tilting frequency of 1 minute) was expected to generate 3.4 dyne/cm² of the maximum shear stress at a higher frequency.

### Induction of in Vitro Ischemic Condition

To induce ischemia, the chips were placed in the incubation chamber of an EVOS fl auto imaging system with 2% O₂ and 5% CO₂ for 24 hours. Before the chips were incubated in the hypoxic chamber, the culture media was replaced with serum- and glucose-free DMEM (Gibco, Cat. No.: 11966025) that had been flushed with nitrogen gas for one minitue and stored at the same hypoxic condition (2% O₂ and 5% CO₂) for overnight before use. There was no flow during the ischemic period. The samples in a normoxic condition were cultured an incubator with 5% CO₂ and atmospheric O₂ concentration (~20%).

### Measurement of Cell Viability and Cytotoxicity

Cell viabilities and cytotoxicity were measured using Live/Dead™ Viability kit (ThermoFisher Scientific, Cat. No.: L3224) and LDH-Cytox Assay™ kit (BioLegend, Cat. No.: 426401), respectively. Cell viability was calculated as the number of viable cells divided by the total number of cells. The relative cytotoxicity of the ischemia was calculated based on the optical densities (OD) read at a wavelength of 490 nm as follows:

$$C y t o t o x i c i t y = \frac{O {D}_{i s c h e m i c s a m p l e} - O {D}_{n o r m o x i c s a m p l e}}{O {D}_{w h o l e c e l l l y s a t e} - O {D}_{n o r m o x i c s a m p l e}}$$

### Functional Characterization of the Reconstructed BBB

### Evaluation of BBB as a Physically Intact Barrier

To evaluate the physical intactness of the formed BBB in a microfluidic chip, a FITC-conjugated dextran (70 kDa and 4 kDa) was injected into the ‘blood-side’ channel and diffusion across it was monitored. Fluorescence images (at 488 nm) were obtained at different time points in one hour after the probe injection and the fluorescence intensities were measured with ImageJ (NIH). The permeability coefficients were calculated by the equation below.

$${P}_{a p p} = \frac{1}{A ⋅ {C}_{0}} ⋅ \frac{d Q}{d t} ≅ \frac{1}{A ⋅ \left({\overline{I}}_{o}^{b l o o d} - {\overline{I}}_{o}^{b r a i n}\right)} ⋅ \frac{{V}^{b r a i n} ⋅ Δ {\overline{I}}^{b r a i n}}{Δ t}$$

A = the surface area of the membrane, C₀ = initial concentration on the donor side,

$$\frac{d Q}{d t} =$$

the transport rate.
V^{brain}= hydrogel volume in the ‘brain’ channel, I̅^{brain}= mean fluorescence intensity in the ‘brain’ channel,
I̅^{blood}= mean fluorescence intensity in the ‘blood-side’ channel, Iₒ═ initial fluorescence intensity

This equation assumes that flux across the imaging boundary is negligible, and transendothelial flux is constant. In the chip described herein, these assumptions were safely met for the time intervals (Δt) shorter than 15 minutes; i.e. there were no significant difference between Pₐₚₚ calculated with 5, 10 and 15 minutes of Δt (n = 5, p-value > 0.05). Δt was set to 10 minutes. The Pₐₚₚ of the BBB

$$\left({P}_{a p p}^{e n d o}\right)$$

was calculated based on the Pₐₚₚ of the whole barrier

$$\left({P}_{a p p}^{w h o l e b a r r i e r}\right)$$

and the endothelium itself

$$\left({P}_{a p p}^{h y d r o g e l}\right)$$

using the following equation.

$$\frac{1}{{P}_{a p p}^{w h o l e b a r r i e r}} = \frac{1}{{P}_{a p p}^{h y d r o g e l}} + \frac{1}{{P}_{a p p}^{e n d o}}$$

### Evaluation of BBB as a Bio-Chemically Intact Barrier

Whether the formed endothelium could isolate the neural cells in the ‘brain’ channel from the serum in the ‘blood-side’ channel was examined. First, the serum-containing medium of ECM and PM (9:1, v/v) containing 10% FBS was added into the ‘blood-side’ channel. After incubation for 24 h, microglia behavior were studied by immunostaining them with its reactive marker, ionized calcium-binding adapter molecule 1 (IBA-1), and its activation marker, differentiation 68 (CD68).

### Evaluation of BBB as a Cell-Selective Barrier to Invading Cells

Whether the reconstructed BBB could show distinct responses to different types of invading cells was examined; two types of human breast cancer cell lines were tested for this purpose: MB-231 and its brain metastatic derivative population, MB-231Br. The cells were prestained with Vybrant™ DiO cell-labeling solution (Invitrogen, Cat. No.: V22886). For prestaining, cells were incubated with staining medium (5 µL labelling solution per 1 mL culture medium) for 20 min in a cell culture incubator and washed with sterile PBS (Phosphate-buffered saline, pH = 7.4) for 3 times. And 10 µL of the cells were injected into the ‘blood-side’ channel at 1 × 10⁶ cells/mL density. The chip was tilted slightly so that the cells could pile up on the hydrogel border. Images were taken 1 day or 10 days after the cancer cell injection using an EVOS fl auto imaging system (Life Technologies).

### Neuronal Degeneration Staining

Neuronal degeneration induced by ischemia was studied using Fluoro-Jade C (FJC) Staining Kit according to the manufacture’s protocol (Biosensis, biosensis® Ready-to-Dilute (RTD) TM Fluoro-Jade^{®} C Staining Kit, Cat. No.: TR-100-FJ) with some modifications. To make sure staining solutions diffuse well into the hydrogel in the middle channel of the chips where neurons were growing, incubation time of staining solutions on the protocol were tripled. After staining, samples were washed at least three times with PBS. For each wash, the incubation time was 5 - 10 min. Images were obtained with EVOS fl microscope (Life Technologies).

### Immunocytochemistry (ICC) of NVU Chips

For fixation of cells in a NVU chip, 50 - 60 µL of 4% paraformaldehyde (PFA) was added as droplets onto the inlet of each channel and kept in the channels for at least 30 min. The fixed hydrogel matrix was gently washed by adding 30 - 40 µL of PBS drops onto the inlet of each channel. This washing was repeated at least 5 times. Then, the cells in the NVU chip were permeabilized in 0.1% Triton X-100 in PBS for 10 - 15 min. The permeabilized cells were washed with PBS for 5 times and then blocked with 5% normal donkey serum in PBST (0.05 % Tween 20 in PBS) for 40 min to 1 hr. The blocked cells were incubated with primary antibodies (30 - 40 µL per channel) at least overnight at 4° C. The dilution ratio of primary antibodies was as follows: sheep polyclonal anti-human CD31/PECAM-1 (R&D Systems, Cat. No.: AF806, 1:20), rabbit polyclonal anti-human GFAP (Sigma, Cat. No.: G9269, 1:100), chicken polyclonal anti-human GFAP (Synaptic Systems, Cat. No.: 173006, 1:500), rabbit polyclonal anti-human AQP4 (Novus Biologicals, Cat. No.: NBP1-87679, 1:2000), mouse monoclonal anti-human ZO-1 (Invitrogen, Cat. No.: 339100, 1:100), rabbit polyclonal anti-human von Willebrand Factor (vWF, Sigma, Cat. No.: F3520, 1:200), mouse monoclonal anti-human vWF (Sigma, Cat. No.: AMAB90931, 1:500), mouse monoclonal anti-human podoplanin (PDPN) (E-1) (Santa Cruz Biotechnology, Cat. No.: SC376695, 1:100), rabbit polyclonal anti-human Synapsin ½ (Synaptic System, Cat. No.: 106003, 1:1,000), chicken polyclonal anti-human MAP2 (Abcam, Cat. No.: ab5392, 1:10,000), goat polyclonal anti-human IBA-1 (Abcam, Cat. No.: ab5076, 1:200), rabbit polyclonal anti-human IL-1β (Abcam, Cat. No.: ab9722, 1:100), mouse monoclonal anti-human CD68 (Bio-Rad, Cat. No.: MAC5709, 1:100), rabbit monoclonal anti-human CD44 (Invitrogen, Cat. No.: 19H8L4, 1: 500), mouse monoclonal anti-human CD34 (Life technology, Cat. No.: BI-3C5, 1:250), mouse monoclonal anti-human Nestin (ThermoFisher, Cat. No.: MA1-5840, 1: 250), rabbit monoclonal anti-human PDGFR β (Cell Signaling, Cat. No.: 3169, 1:100), and mouse monoclonal anti-human HIF-1 α (Abcam, Cat. No.: ab6066, 1:200). To prevent dryness during the primary antibody incubation, the plates containing the chips were humidified with distilled water. Incubated samples were washed with blocking solution for 5 times, and then various secondary antibodies (Jackson ImmunoResearch Laboratories) including DyLight405 anti-rabbit, Alexa Fluor 488 anti-chicken, mouse, or rabbit, Alexa Fluor 594 anti-mouse, rabbit, or sheep, and Alexa Fluor 647 anti-mouse, rabbit, or sheep were added to samples at a dilution of 1:500 at room temperature for at least 2 h. The immunostained slides were mounted with ProLong Diamond antifade reagent (ThermoFisher) and cured for 24 h. To prevent collapsing of the fixed hydrogel structure, all buffers including PFA, PBS, and blocking solutions were not fully removed from the outlet reservoir throughout the whole procedure of ICC.

### 3D Images of BBB

Fluorescent images of immunostained-BBB structures were acquired at 10× and 20× magnifications with a Zeiss LSM 880 confocal microscope. The NVU chip was scanned at different focal planes ranging from Z = 0 to 100 µm with 8 - 10 µm intervals. For 3D reconstruction of images, the 3D Viewer in Plugins of ImageJ was used.

### Calcium Imaging and Analysis

To record the cytosolic calcium oscillation in the neurons differentiated from iPSC-derived NPC, the iPSC-derived NPC were pre-stained with DiL (1,1′-Dioctadecyl-3,3,3',3′-Tetramethylindocarbocyanine Perchlorate, Molecular probes, Cat. No.: D282) before incorporating with other cells, as described in the process of Reconstruction of brain tissue and BBB. One hour prior to calcium imaging, 5 µM Fluo-4 AM (Thermo Fisher Scientific, Cat. No.: F14201) was added to the inlets of the chips. Spontaneous calcium oscillations were observed in DiL labeled cells using a confocal microscope (Carl Zeiss, LSM 710, Göttingen, Germany) under 37° C. and 5 % CO₂. Calcium signaling was recorded in time-lapse video recording mode at a speed of 2 sec/frame for 10 minutes. The recorded images were analyzed using ImageJ software with Time Series Analyzer V3 plugin. Calcium dynamics in each DiL labeled cell was traced with ROI (Region of Interest) analysis, and the parameters of calcium oscillation in each cell, such as frequency and amplitude (ΔF/F₀) were calculated using a custom-made script written in IDL (Interactive digital language). Extracellular background signal was subtracted from the traced calcium signals, which in turn were normalized to the intracellular basal line (F₀).

### Real-Time Quantitative PCR

The gene expression pattern of the entire cell population was evaluated. More than six chips from each experimental condition were used to extract RNA for analysis. Cells from different batches were used to ensure cell numbers sufficient to prepare multiple chips simultaneously. The culture medium in both of the side channels was removed and replaced with fresh PBS. The process was repeated twice with 5 min incubation in between. After removal of PBS, 50 uL of RLT buffer plus lysis buffer (Qiagen, Cat. No.: 1053393) was injected into both of the side channels and incubated for 5 min. Thorough pipetting was needed in order to harvest all types of cells in the chip, especially those in the ‘brain’ channel. Total RNA was then extracted using RNeasy Mini Kit according to the manufacturer’s protocol (Qiagen, Cat. No.: 74104). RNA quality and concentration were determined by Agilent 210 Bioanalyzer. The RNA amount obtained from each chip was as follows for each experimental condition: a chip with 10 days under normoxia generated about 250 ng; the same condition followed by 24 hours of ischemia generated about 130 ng; a chip with 18 days under normoxia, used as the control group in neurorestorative efficacy evaluation, about 400 ng; a chip with reperfusion only, about 200 ng; a chip with transplanted stem cells, about 350 ng to 750 ng depending on the stem cell type. Total RNA was reverse transcribed to cDNA using a high-capacity cDNA reverse transcription kit (Applied Biosystems, Cat. No.: 4368814). Real-time qPCR was performed in a StepOnePlus real-time PCR system (Applied Biosystems) using SsoAdvanced Universal SYBR Green Supermix (Bio-Rad, Cat. No.: 1725272A) to quantify the expression levels of the genes of interest. qPCR amplification was achieved with 40 cycles of 30 s at 95° C., 15 s at 95° C., and 50 s at 65° C. The signal was distinguished from noise using StepOnePlus real-time PCR software and further checked manually to ensure the obtained Ct values had indeed come from real signals. Customized qPCR plates were designed and fabricated by Sciencell. Neurogenesis qPCR plates were purchased from Qiagen. 12 genes with well-known ischemic behaviors were first chosen and confirmed the reproducibility of their expression by triplicate. The expression of the final 123 genes were measured with more than six independent chips for each experimental condition.

### Evaluation of the Neurorestorative Potential of Stem Cells

The neurorestorative potential of various types of stem cells was examined. 24 hours after the ischemic insult, the serum- and glucose-free DMEM medium was replaced with NDM/AM (the mixed medium of NDM, serum-free AM, and ACM at the ratio of 8:4:1 (v/v/v)) in the ‘CSF-side’ channel. Then the stem cells were collected at 5 × 10⁶ cells/mL density in ECM/PM (the mixed medium of serum-containing ECM and PM at the ratio of 9:1 (v/v)) and injected 10⁴ cells into the ‘blood-side’ channel. This setting represented the intra-vascular route for stem cell transplantation, the most widely used route for stem cell therapy. The stem cells were incubated for 3 hours to allow for cell adhesion and floating cells were gently washed away with fresh ECM/PM. Medium in both of the side channels was changed every day for 7 more days before further analysis.

### Tracking Stem Cells

In order to track stem cell behaviors in the chip, a lentivirus vector carrying green fluorescent protein (GFP) was used to transfect stem cells hNPC, hNSC (NR1), hAMSC, hBMSC, and hEPC. A pre-staining method was used for hHSC because the virus transfection efficiency of hHSC was not sufficient. Ready-to-use GFP lentiviral particles were purchased from GenTarget Inc (San Diego, CA, USA) and used according to the manufacturer’s protocol with some modification. More specifically, cells were cultured in a 48-well plate until the confluency reached 50% to 75%. Cell culture medium was removed before transduction and 0.25 mL of fresh medium and 15 µL of virus solution were added to each well. Cells were cultured in a cell culture incubator for 2 - 3 days without medium change in between to achieve desirable transduction efficiency. hHSC was prestained with Vybrant™ DiO cell-labeling solution (Invitrogen, Cat. No.: V22886). hHSCs at 1 × 10⁶ cells/mL were incubated with staining medium (10 µL labelling solution per 1 mL culture medium) in a 96-well plate for 20 min in a cell culture incubator and washed with sterile PBS (Phosphate-buffered saline, pH = 7.4) for 3 times for use. After injecting each stem cell into the ‘blood-side’ channel of a chip following ischemic insult, images were taken every other day for up to 7 days to track stem cell infiltration. At the end of the 7 days, stem cells were immunostained for sternness markers (Nestin for hNPC and hNSC, CD44 for hBMSC and hAMSC, and CD34 for hEPC and hHSC) and differentiation markers (MAP2 for neurogenesis, GFAP for gliagenesis, and von Willebrand Factor for vasculogenesis). The extravasation extent of both cancer and stem cells was quantified by image scoring (ImageJ, NIH).

### Statistical Methods

Every independent experiment was repeated at least three times, and the results were presented as the mean ± standard deviation (S.D.). For the quantitative analysis of fluorescence images, at least three images were obtained from different samples and used an image analysis software, ImageJ (NIH), to quantitatively analyze the aspects of interest. Statistical significance was evaluated using one-sided Student’s t-Test for two group comparisons and one way ANOVA with Bonferroni-Holm post hoc test for multiple group comparison (Daniel’s XL Toolbox). P-values less than 0.05 were considered significant.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Pat. Application No. 63/013,903 are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A microfluidic chip, comprising:
a planar surface;
a first channel formed on the planar surface and having a first volume defined by a first width, a first height, and a first length, the first channel extending in a first direction; and
a second channel formed on the planar surface adjacent to the first channel, the second channel having a second volume defined by a second width, a second height, and a second length, the second channel extending in the first direction, the second height being greater than the first height;
wherein the first channel is in fluid communication with the second channel through a first opening that extends along at least a portion of the first length, wherein the first opening extends from the planar surface to the first height; and
wherein the first height and the second height are selected such that surface tension of a liquid added to either the first channel or the second channel provides a non-physical microfluidic barrier that selectively limits passage of the liquid through the first opening.

2. The microfluidic chip of claim 1, further comprising:
a third channel formed on the planar surface adjacent to the first channel, the third channel having a third volume defined by a third width, a third height, and a third length, the third channel extending in the first direction, the third height being greater than the first height;
wherein the third channel is in fluid communication with the first channel through a second opening that extends along at least a portion of the first length, wherein the second opening extends from the planar surface to the first height; and
wherein the first height and the third height are selected such that surface tension of a liquid added to the first channel or the third channel provides a non-physical microfluidic barrier that selectively limits passage of the liquid through the second opening .

3. The microfluidic chip of claim 1, further comprising:
a third channel formed on the planar surface adjacent to the second channel, the third channel having a third volume defined by a third width, a third height, and a third length, the third channel extending in the first direction, the third height being less than the second height;
wherein the third channel is in fluid communication with the second channel through a second opening that extends along at least a portion of the second length, wherein the second opening extends extending from the planar surface to the third height; and
wherein the second height and the third height are selected such that surface tension of a liquid added to the second channel or the third channel provides a non-physical microfluidic barrier that selectively limits passage of the liquid through the second opening .

4. A microphysiological system, comprising:
the microfluidic chip of claim 1; and
an extracellular matrix for use within the first channel, wherein a side wall of the extracellular matrix when added to the first channel and gelled extends across the first opening and forms a further barrier between the first channel and the second channel.

5. The microphysiological system of claim 4, further comprising an epithelial barrier arranged in the first opening between the first channel and the second channel.

6. (canceled)

7. The microphysiological system of claim 5, wherein the epithelial barrier comprises endothelial cells.

8. (canceled)

9. The microphysiological system of claim 7, wherein the endothelial cells comprise Brain Microvascular Endothelial Cells (BMEC).

10. (canceled)

11. The microphysiological system of claim 4 , further comprising pericytes in the second channel.

12. The microphysiological system of claim 4 , further comprising first cells in the extracellular matrix in the first channel.

13. The microphysiological system of claim 12, wherein the first cells are neural cells.

14. The microphysiological system of claim 13, wherein the neural cells comprise human induced pluripotent stem cell-derived neural progenitor cells, astrocytes, microglia, or combinations thereof.

15. The microphysiological system of claim 12, wherein the first cells are cardiac cells, skeletal muscle cells, hepatic cells, renal cells, bone cells, skin cells, esophageal cells, intestinal cells, gastric cells, colon cells, lung cells, or pancreatic cells.

16. The microphysiological system of claim 4 , wherein the extracellular matrix comprises a hydrogel.

17. The microphysiological system of claim 16, wherein the hydrogel comprises basement membrane extract (BME).

18. (canceled)

19. The microphysiological system of claim, further comprising
a first media within the second channel;
a third channel formed on the planar surface adjacent to the first channel, wherein the third channel has a third volume defined by a third width, a third height, and a third length,
wherein the third channel extends in the first direction, and wherein the third height is greater than the first height,
wherein the third channel is in fluid communication with the first channel through a second opening that extends along at least a portion of the first length, wherein the second opening extends from the planar surface to the first height; and
wherein the first height and the third height are selected such that surface tension of a liquid added to the first channel or the third channel provides a non-physical microfluidic barrier that selectively limits passage of the liquid through the second opening; and
a second media confined within the third channel.

20. The microphysiological system of claim 19 , wherein the third height is less than the second height, and the microphysiological system further comprises a second extracellular matrix confined within the third volume of the third channel.

21. The microphysiological system of claim 4 , wherein the microphysiological system is a vascularized tissue model, wherein the gelled extracellular matrix in the first channel comprises first cells, and the second channel contains endothelial cells and pericyte cells in a liquid medium.

22. The microphysiological system of claim 21, wherein the vascularized tissue model is a blood brain barrier model, wherein the first cells are neural cells including astrocytes.

23. The microphysiological system of claim 21, wherein the vascularized tissue model is a cardiac model wherein the first cells are cardiac cells, a skeletal muscle model wherein the first cells are muscle cells, a liver model wherein the first cells are liver cells, a kidney model wherein the first cells are kidney cells, a bone model wherein the first cells are bone cells, a skin model wherein the first cells are skin cells, an esophageal model wherein the first cells are esophageal cells, a gastric model wherein the first cells are stomach cells, a colon model wherein the first cells are colon cells, an intestinal model wherein the first cells are intestinal cells, a lung model wherein the first cells are lung cells, or a pancreatic model wherein the first cells are pancreatic cells.

24. The microphysiological system of claim 4, wherein
the extracellular matrix is added to the first channel in liquid form and is then gelled.

25-26. (canceled)

27. A method for screening a therapeutic agent, the method comprising:
depositing the therapeutic agent in the second channel of the microfluidic chip of a microphysiological system of claim 4; and
imaging the microfluidic chip.

28. The method of claim 27, wherein the therapeutic agent comprises a stem cell, a small molecule, or a peptide.

29. (canceled)
